SEQUENCE LISTING (1 sequence)

SEQ ID NO: 1 - protein, 2620 aa, Homo sapiens
MPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTVSIGGHGLGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

(19) United States
(12) Patent Application Publication
(10) Pub. No.: US 2025/0295643 A1
(43) Pub. Date: Sep. 25, 2025

(54) METHODS OF TREATING EPILEPSY

(71) Applicant: YALE UNIVERSITY, New Haven, CT (US)

(72) Inventors: Angelique Bordey, Guilford, CT (US); Longbo Zhang, Guilford, CT (US)

(21) Appl. No.: 18/967,510

(22) Filed: Dec. 3, 2024

Related U.S. Application Data
(63) Continuation of application No. 17/430,549, filed on Aug. 12, 2021, now Pat. No. 12,186,307.
(60) Provisional application No. 62/805,151, filed on Feb. 13, 2019.
(60) Provisional application No. 62/805,792, filed on Feb. 14, 2019.
(60) Provisional application No. 62/810,094, filed on Feb. 25, 2019.
(60) Provisional application No. 62/958,947, filed on Jan. 9, 2020.

(51) Int. Cl. A61K 31/438; A61K 31/192; A61P 25/08; A61P 25/28; G01N 33/68

(52) U.S. Cl. CPC A61K 31/438; A61K 31/192; A61P 25/08; A61P 25/28; G01N 33/6896; G01N 2333/4712; G01N 2333/70571; G01N 2800/2821; G01N 2800/50

(57) ABSTRACT

In various aspects and embodiments the invention provides a method of treating epilepsy in a subject in need thereof, the method comprising providing to the subject an effective amount of an FLNA modulator. In various embodiments, the FLNA modulator is PTI-125 or kartogenin. In various embodiments, the epilepsy is epilepsy associated with focal cortical dysplasia (FCD) type II or tuberous sclerosis complex (TSC).

### CROSS-REFERENCE TO RELATED APPLICATIONS

The present application is a continuation of, and claims priority to, U.S. patent application Ser. No. 17/430,549, filed Aug. 12, 2021, issued as U.S. Pat. No. 12,186,307, which is a 35 U.S.C. § 371 national phase application of, and claims priority to, International Application No. PCT/US2020/018136 filed Feb. 13, 2020, which claims priority under 35 U.S.C. § 119 (e) to U.S. Provisional Patent Application Nos. 62/805,151 filed Feb. 13, 2019, 62/805,792 filed Feb. 14, 2019, 62/810,094 filed Feb. 25, 2019, and 62/958,947, filed Jan. 9, 2020, all of which applications are incorporated herein by reference in their entireties.

### STATEMENT REGARDING FEDERALLY SPONSORED RESEARCH OR DEVELOPMENT

This invention was made with government support under NS111074 and NS093704 awarded by the National Institutes of Health. The government has certain rights in the invention.

### SEQUENCE LISTING

The XML text file named “047162_7218US2_SequenceListingST26.xml” created on Dec. 3, 2024, comprising 4,469 bytes, is hereby incorporated by reference in its entirety.

### BACKGROUND OF THE INVENTION

Epilepsy occurs in 80-90% of individuals with cortical malformations and is often refractory to treatments. The largest subset of cortical malformations shares similar molecular, histopathological, and clinical features, suggesting a common mechanism through which they contribute to epileptogenesis. These are focal cortical dysplasia (FCD) type II, STRADalpha deficiency, and tuberous sclerosis complex (TSC). Somatic mutations in these neurodevelopmental disorders occur during embryonic life and lead to hyperactivity of the mTOR complex 1 (mTORC1) and the formation of focal cortical malformations. Treatment options are limited to surgical resection of the focal cortical malformations or treatment with everolimus. Most patients will try every other drug available, but without success. Many patients cannot undergo surgery because the malformation(s) are not accessible or too numerous. In addition, surgery is an invasive and traumatic experience for patients and is not always fully successful as 30-40% of the patients will not properly manage their seizures post-surgery. Everolimus clinical trial reported that everolimus (at the highest dose) was efficient at reducing seizure frequency in a subset of patients (40%), but had side-effects and 60% of the patients saw no improvements. There is thus a critical need to find a better treatment option to reduce or eliminate their seizures in individuals with TSC or FCDII. In addition, recurrent seizures are accompanied by significant comorbidity, including neurocognitive and psychological deficits as well as poor quality of life. Preventing seizures from occurring or reducing their frequency would significantly improve the life of individuals with the disease as well as that of their caregiver. There is a need in the art for novel methods for treating epilepsy. This disclosure addresses that need.

### SUMMARY OF THE INVENTION

In one aspect, the invention provides a method of treating epilepsy in a subject in need thereof, the method comprising providing to the subject an effective amount of an FLNA modulator.

In another aspect, the invention provides a method of treating focal cortical dysplasia (FCD) type II or tuberous sclerosis complex (TSC) in a subject in need thereof, the method comprising providing to the subject an effective amount of PTI-125.

In yet another aspect, the invention provides of a method of inhibiting hyperphosphorylation of the tau protein that comprises the steps of administering to cells of the central nervous system in recognized need a FLNA-binding effective amount of kartogenin or a pharmaceutically acceptable salt.

In yet another aspect, the invention provides a method for determining the likelihood of a living patient having Alzheimer's disease pathology (AD pathology) comprising the steps of
a) determining the amount of one or more of a protein-protein complex selected from the group consisting of i) α7nAChR/FLNA, ii) TLR4/FLNA and iii) α7nAChR/AB present in a first portion of a lymphocyte preparation from said living patient:
b) determining the amount of said one or more of i) α7nAChR/FLNA, ii) TLR4/FLNA and iii) α7nAChR/AB present as a protein-protein complex in a second portion of said lymphocyte preparation, said second portion of said lymphocyte portion further containing admixed therein a FLNA binding-effective amount of kartogenin or a pharmaceutically acceptable salt thereof; and
c) comparing the values so determined, whereby a determined amount of said one or more of i) α7nAChR/FLNA, ii) TLR4/FLNA and iii) α7nAChR/Aβ present as a protein-protein complex in a second portion of said lymphocyte preparation that is significantly decreased in the presence of kartogenin or pharmaceutically acceptable salt thereof indicates that the patient had AD pathology at the time the body sample was taken, whereas no significant difference between the two determined values indicates that the patient was free of AD pathology at the time the body sample was taken.

In certain embodiments, the FLNA modulator is PTI-125.

In certain embodiments, the FLNA modulator is kartogenin

In certain embodiments, the epilepsy is intractable epilepsy.

In certain embodiments, the epilepsy is associated with focal cortical dysplasia (FCD) type II or tuberous sclerosis complex (TSC) or hemimegalencephaly.

In certain embodiments, the FLNA modulator is formulated in a pharmaceutical composition comprising at least one pharmaceutically acceptable excipient.

In certain embodiments, the subject is a mammal. In certain embodiments, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A-1I depict that FLNA expression is increased in cortices of patients with FCDII. (FIG. 1A) MRI scans of a 2-year and a 5-year old patient with FCDII with seizures. The arrow points to the FCM. (FIG. 1B and FIG. 1C) Images of 3pproxim-S6 (FIG. 1B) and FLNA (FIG. 1C) staining in hematoxylin-stained sections from the FCM in the brain of patients whose scans are shown in (FIG. 1A). Arrows point to FLNA-positive balloon cells and dysmorphic neurons. (FIG. 1D and FIG. 1E) Co-immunostaining for FLNA and a marker of cytomegalic neurons, SMI-311 and DAPI counterstain in human FCDII tissue. (FIG. 1F) Magnification of SMI311 or FLNA staining with DAPI from images in the white square in FIG. 1D and FIG. 1E. (FIG. 1G) Quantification of FLNA staining in SMI-311-positive neurons relative to surrounding SMI-311-negative cells. Only SMI-311-negative cells with visible FLNA staining (to outline the cell body) were used for quantification. (FIG. 1H and FIG. 1I) Immunoblots of FLNA and tubulin from resected FCDII tissue (FIG. 1H) and quantification from 12 patients with FCDII (I). The numbers correspond to those of the patients listed in Tables 1 and 2. FLNA and tubulin size were 280 and 55 kDa, respectively. Unpaired Student t-test. Data are mean±SEM. N number are listed in Table 3. Scale bars: 100 μm.

FIG. 2A-2H depict that FLNA expression is increased in mouse cortices containing Rheb^{CA}-induced FCM. (FIG. 2A) FLNA (280 kDa), pS6 (32 kDa), S6 (32 kDa), and GAPDH (37 kDa) immunoblots in cultured neurons expressing plasmids encoding GFP. mTORC1 mutants, or Rheb^{CA}. (FIG. 2B and FIG. 2C) Quantification of the blots show in FIG. 1A. (FIG. 2D) Diagram of the experimental procedure for generating FCM in mice followed by video-EEG recordings and immunoblotting or immunochemistry. (FIG. 2E and FIG. 2F) FLNA immunostaining in sections from mouse FCM (FIG. 2E) and quantification in GFP-positive Rheb^{CA}-expressing cells relative to surrounding GFP-negative cells (FIG. 2F). Scale bar: 100 μm. (FIG. 2G) FLNA and GAPDH immunoblots. Ipsi: ipsilateral and Contra: contralateral. (FIG. 2H) FLNA/GAPDH quantification in FCM-containing cortices relative to contralateral cortices in mice electroporated with Rheb^{CA}. One way ANOVA (FIG. 2B and FIG. 2C), unpaired and paired Student t-test (FIG. 2F and FIG. 2H. respectively). Data are mean±SEM. N number are listed in Table 3.

FIG. 3A-3H depict that normalizing the amount of FLNA in dysmorphic neurons of mTORC1-driven FCM partially prevents cytoarchitectural abnormalities and attenuates seizure activity. (FIG. 3A) Images of control or Rheb^{CA}-expressing neurons co-expressing luciferase or Flna shRNA in littermate mice, and quantification of neuronal placement in the different conditions. Scale bar: 250 μm. (FIG. 3B) Images of GFP+ electroporated neurons in different conditions and quantification of soma size. Scale bar: 50 μm. (FIG. 3C) Basal dendrite reconstruction and corresponding Sholl analysis. Scale bar: 50 μm. (FIG. 3D) Bar graphs of the basal total dendrite length (TDL). (FIG. 3E) Diagram of the experimental procedure. (FIG. 3F) Representative EEG traces in a control shRNA seizing mouse. Scales: 200 uV/5 s and 1 s (inset). (FIG. 3G) Frequency of seizures in the Luc or Flna shRNA condition. (FIG. 3H) Phospho-S6 immunostaining and quantification in GFP+ Rheb^{CA}-expressing neurons (normalized to surrounding GFP-neurons) in EEG-recorded mice. Scale bar: 50 μm. Mann Whitney tests (FIG. 3G), one-way ANOVA (FIGS. 3A, 3B, and 3D) and two-way repeated measure ANOVA (FIG. 3C), and Student's t test (FIG. 3H). Data are mean±SEM. N number are listed in Table 3.

FIGS. 4A-4J depict that treatment with a small molecule modulator of FLNA. PTI-125, prior to seizure onset partially prevents cytoarchitectural abnormalities and reduces seizure activity. (FIG. 4A) Diagram of experimental paradigm. (FIG. 4B) Images of neuronal soma following treatment with vehicle or different PTI-125 doses and quantification. Scale bar: 50 μm. (FIG. 4C and FIG. 4D) Reconstructions of basal dendrites (FIG. 4C), Sholl analyses, and basal total dendritic length (TDL. FIG. 4D) under different treatment conditions. (FIG. 4E) Immunoblots of 5pproxim-S6 (pS6), S6 and GAPDH from the cortices of mice treated with vehicle (saline) or PTI-125, and quantification. (FIG. 4F) Diagram of experimental paradigm. (FIG. 4G) Seizure frequency (6 day-long recordings) following vehicle or PTI-125 treatment at 12 mg/kg. (FIG. 4H) Plots of the weight gain during vehicle or PTI-125 treatment from P8-P60. Inset: mean body weight between P52-P60. (FIG. 4I) Quantification of cell misplacement in mice treated with vehicle or PTI-125. (FIG. 4J) Images of phoshpho-S6 (pS6) immunostaining and GFP fluorescence in coronal sections containing Rheb^{CA}-expressing cells (GFP+) and corresponding 5pproxim-S6 (pS6) quantification normalized to the vehicle treated condition. Scale bar: 50 μm. Student t-test (FIG. 4E, FIG. 4H, FIG. 4 I, and FIG. 4J), two-way repeated measure ANOVA (FIG. 4D), Mann Whitney test (FIG. 4G), and one-way ANOVA (FIG. 4B and FIG. 4D). Data are mean±SEM. N number are listed in Table 3.

FIGS. 5A-5K depict that treatment with a small molecule modulator of FLNA, PTI-125, after seizure onset alleviates neuronal dysmorphogenesis and seizure activity. (FIG. 5A) Diagram of experimental paradigm. (FIGS. 5B and 5C) Images of control GFP+ neurons and GFP+ Rheb^{CA}-expressing neurons in mice treated with vehicle (saline) or PTI-125 (FIG. 5b) and quantification of soma sizes (FIG. 5C). Scale bar: 120 μm. (FIG. 5D and FIG. 5E) Sholl analyses and total basal dendritic length (TDL). (FIG. 5F) Heatmap of seizure activity over time (per day) per mouse either treated with vehicle (saline) or PTI-125. (FIG. 5G) Seizure frequency following and during vehicle or PTI-125 treatment from P29 to P54. (FIG. 5H) Diagram of experimental paradigm. (FIG. 5I) Heatmap of seizure activity per individual mouse over time. In the upper heatmap, mice received vehicle injections and were recorded from P61 to P92. In the bottom heatmap, mice received vehicle from P61 to P72 and then PTI-125 until P106. For mice #5, the wires were unplugged for two days leading to loss of recordings that are colored white on the heatmap. (FIG. 5J) Bar graph of the seizure frequency at days (FIG. 5D) 1-5 and D27-31 of recordings (corresponding to P61-65 and P88-92) under continuous vehicle treatment or vehicle-to-PTI-125 treatment. (FIG. 5K) Plots of the seizure frequency prior to and after PTI-125 treatments. One-way ANOVA (FIG. 5C. FIG. 5E, and FIG. 5J), two-way repeated measure ANOVA (FIG. 5D), Mann Whitney tests (FIG. 5G), and Wilcoxon matched pairs test (FIG. 5K). Data are mean±SEM. N number are listed in Table 3.

FIGS. 6A-6C depict that PTI-125 decreases the percentage of seizing mice and seizure frequency. (FIG. 6A) Percentage (%) of seizure-free mice in both conditions. Two mice in vehicle treated condition died of seizures during EEG recordings and were thus not included in FIG. 6B. (FIG. 6B and FIG. 6C) Heatmap of seizure frequency for all conditions (FIG. 6B) and corresponding scatter plot (FIG. 6C). Fisher Exact test (FIG. 6A) and Mann Whitney test (FIG. 6C). Data are mean±SEM.

FIG. 7A-7F depict images of GFP+ neurons in coronal sections from mice electroporated with increasing concentrations of Rheb^{CA}. FIG. 7B depicts plots of the somata size as a function of Rheb^{CA} concentration. FIG. 7C depicts representative EEG recording of seizures in the different Rheb^{CA} condition. FIG. 7D depicts a plot of the seizure frequency (#seizures per day) as a function of the concentration of Rheb^{CA}. FIGS. 7E and 7F depict plots of the seizure frequency (FIG. 7E) and duration (FIG. 7F) for two Rheb^{CA} concentrations. T-test.

FIG. 8 depicts a graph quantifying the effect of kartogenin (KGN) or vehicle on the total neurite length in Rheb^{CA}-expressing neurons cultured for 7 days in vitro (DIV). Unpaired, two-sided t-test P<0.0001. Neurons were treated at the time of plating until harvesting with 5 μM KGN. 20)

FIGS. 9A-9F depict that FLNA expression is increased in cortices of patients with FCDII. (FIG. 9A-FIG. 9D) Images of FLNA and 6pproxim-S6 staining in hematoxylin-stained cortical sections from patients with FCDII. Scale bar: 100 μm. (FIG. 9E and FIG. 9F) Quantification of 6pproxim-S6 and FLNA staining intensity in dysmorphic cells versus surrounding cells. Unpaired Mann-Whitney and Student t-test. Data are mean±SEM. N numbers are listed in Table 3.

FIGS. 10A-10E depict that FLNA expression is increased in cytomegalic cortical neurons of patients with FCDII. (FIG. 10A-FIG. 10E) Images of FLNA and SMI-311 co-staining and DAPI counterstain in cortical sections from patients with FCDII. Scale bars: 140 μm (FIG. 10A, FIG. 10B, and FIG. 10D), 25 μm (FIG. 10C), and 100 μm (FIG. 10E).

FIGS. 11A-11B depict original immunoblots for FLNA in human FCDII samples. (FIG. 11A and FIG. 11B) Filamin A (FIG. 11A) and Tubulin (FIG. 11B) immunoblots on the same membranes from human FCDII (noted F) and control (noted C) cortical tissue. The ladder (from Fisher Scientific, #26617) is shown on the left of the blots. The numbers correspond to those in Tables 1 and 2 (human samples).

FIGS. 12A-12C depict that the quantity of FLNA is not correlated to the type of FCDII or age of seizure onset or epilepsy duration. (FIG. 12A) Bar graphs of FLNA quantity in FCDIIa and FCDIIb. (FIG. 12B and FIG. 12C) Scatter plots of the relative quantity of FLNA (mean control is 1) versus the age of seizure onset (FIG. 12B) or the duration of epilepsy history (FIG. 12C). The numbers correspond to the patient number listed in Table 1.

FIGS. 13A-13B depict that knocking down FLNA does not affect the degree of S6 phosphorylation. (FIG. 13A) Immunoblots for the proteins listed on the left in Neuro2A cells transfected with either luciferase or FLNA shRNA. (FIG. 13B) Quantification of FLNA/ERK and pS6/S6 quantities in both transfection conditions. Student t-test. Data are mean±SEM.

FIGS. 14A-14B depict that delineation of layer 2/3 and layer 5 neurons using ER81 immunostaining. (FIG. 14A and FIG. 14B) Immunostaining for ER81 (pseudo-colored red), a marker of layer 5 neurons, and GFP fluorescence in the anterior cingulate cortex from mice electroporated with GFP (+tdTomato) (FIG. 14A) or GFP+ Rheb^{CA} (FIG. 14B) at E15. The dotted line delineates the boundary between layer 2/3 and layer 5.

FIGS. 15A-15C depict single cell labeling for dendrite (sholl) analysis. (FIG. 15A) GFP and DsRed fluorescence from pCAG-GFP and pCALNL-DsRed (inducible vector) and pCAG-Cre. (FIG. 15B) DsRed fluorescence in black and white. (FIG. 15C) Image of Sholl reconstruction of the basal dendrites of the neurons shown in FIG. 15B.

FIGS. 16A-16D-2 depict the original immunoblots. The ladder (from Fisher Scientific. #10748010) is shown on the left of each blot.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, “filamin A” or “FLNA” refers to the protein for which the human homolog has the amino acid sequence:

```
SEQ ID NO: 1
      10 20 30 40
MPATEKDLAE DAPWKKIQQN TFTRWCNEHL KCVSKRIANL
                50 60 70 80
QTDLSDGLRL IALLEVLSQK KMHRKHNQRP TFRQMQLENV
            90 100 110 120
SVALEFLDRE SIKLVSIDSK AIVDGNLKLI LGLIWTLILH
        130 140 150 160
YSISMPMWDE EEDEEAKKQT PKQRLLGWIQ NKLPQLPITN
        170 180 190 200
FSRDWQSGRA LGALVDSCAP GLCPDWDSWD ASKPVTNARE
    210 220 230 240
AMQQADDWLG IPQVITPEEI VDPNVDEHSV MTYLSQFPKA
              250 260 270 280
KLKPGAPLRP KLNPKKARAY GPGIEPTGNM VKKRAEFTVE
            290 300 310 320
TRSAGQGEVL VYVEDPAGHQ EEAKVTANND KNRTFSVWYV
        330 340 350 360
PEVTGTHKVT VLFAGQHIAK SPFEVYVDKS QGDASKVTAQ
      370 380 390 400
GPGLEPSGNI ANKTTYFEIF TAGAGTGEVE VVIQDPMGQK
    410 420 430 440
GTVEPQLEAR GDSTYRCSYQ PTMEGVHTVH VTFAGVPIPR
              450 460 470 480
SPYTVTVGQA CNPSACRAVG RGLQPKGVRV KETADFKVYT
            490 500 510 520
KGAGSGELKV TVKGPKGEER VKQKDLGDGV YGFEYYPMVP
          530 540 550 560
GTYIVTITWG GQNIGRSPFE VKVGTECGNQ KVRAWGPGLE
      570 580 590 600
GGVVGKSADF VVEAIGDDVG TLGFSVEGPS QAKIECDDKG
    610 620 630 640
DGSCDVRYWP QEAGEYAVHV LCNSEDIRLS PFMADIRDAP
              650 660 670 680
QDFHPDRVKA RGPGLEKTGV AVNKPAEFTV DAKHGGKAPL
            690 700 710 720
RVQVQDNEGC PVEALVKDNG NGTYSCSYVP RKPVKHTAMV
          730 740 750 760
SWGGVSIPNS PFRVNVGAGS HPNKVKVYGP GVAKTGLKAH
      770 780 790 800
EPTYFTVDCA EAGQGDVSIG IKCAPGVVGP AEADIDFDII
    810 820 830 840
RNDNDTFTVK YTPRGAGSYT IMVLFADQAT PTSPIRVKVE
              850 860 870 880
PSHDASKVKA EGPGLSRTGV ELGKPTHFTV NAKAAGKGKL
            890 900 910 920
DVQFSGLTKG DAVRDVDIID HHDNTYTVKY TPVQQGPVGV
      930 940 950 960
NVTYGGDPIP KSPFSVAVSP SLDLSKIKVS GLGEKVDVGK
        970 980 990 1000
DQEFTVKSKG AGGQGKVASK IVGPSGAAVP CKVEPGLGAD
    1010 1020 1030 1040
NSVVRFLPRE EGPYEVEVTY DGVPVPGSPF PLEAVAPTKP
            1050 1060 1070 1080
SKVKAFGPGL QGGSAGSPAR FTIDTKGAGT GGLGLTVEGP
          1090 1100 1110 1120
CEAQLECLDN GDGTCSVSYV PTEPGDYNIN ILFADTHIPG
        1130 1140 1150 1160
SPFKAHVVPC FDASKVKCSG PGLERATAGE VGQFQVDCSS
    1170 1180 1190 1200
AGSAELTIEI CSEAGLPAEV YIQDHGDGTH TITYIPLCPG
    1210 1220 1230 1240
AYTVTIKYGG QPVPNFPSKL QVEPAVDTSG VQCYGPGIEG
            1250 1260 1270 1280
QGVFREATTE FSVDARALTQ TGGPHVKARV ANPSGNLTET
          1290 1300 1310 1320
YVQDRGDGMY KVEYTPYEEG LHSVDVTYDG SPVPSSPFQV
        1330 1340 1350 1360
PVTEGCDPSR VRVHGPGIQS GTTNKPNKFT VETRGAGTGG
      1370 1380 1390 1400
LGLAVEGPSE AKMSCMDNKD GSCSVEYIPY EAGTYSLNVT
    1410 1420 1430 1440
YGGHQVPGSP FKVPVHDVTD ASKVKCSGPG LSPGMVRANL
  1450 1460 1470 1480
PQSFQVDTSK AGVAPLQVKV QGPKGLVEPV DVVDNADGTQ
          1490 1500 1510 1520
TVNYVPSREG PYSISVLYGD EEVPRSPFKV KVLPTHDASK
        1530 1540 1550 1560
VKASGPGLNT TGVPASLPVE FTIDAKDAGE GLLAVQITDP
      1570 1580 1590 1600
EGKPKKTHIQ DNHDGTYTVA YVPDVTGRYT ILIKYGGDEI
    1610 1620 1630 1640
PFSPYRVRAV PTGDASKCTV TVSIGGHGLG AGIGPTIQIG
            1650 1660 1670 1680
EETVITVDTK AAGKGKVTCT VCTPDGSEVD VDVVENEDGT
          1690 1700 1710 1720
FDIFYTAPQP GKYVICVRFG GEHVPNSPFQ VTALAGDQPS
        1730 1740 1750 1760
VQPPLRSQQL APQYTYAQGG QQTWAPERPL VGVNGLDVTS
      1770 1780 1790 1800
LRPFDLVIPF TIKKGEITGE VRMPSGKVAQ PTITDNKDGT
    1810 1820 1830 1840
VTVRYAPSEA GLHEMDIRYD NMHIPGSPLQ FYVDYVNCGH
            1850 1860 1870 1880
VTAYGPGLTH GVVNKPATFT VNTKDAGEGG LSLAIEGPSK
          1890 1900 1910 1920
AEISCTDNQD GTCSVSYLPV LPGDYSILVK YNEQHVPGSP
        1930 1940 1950 1960
FTARVTGDDS MRMSHLKVGS AADIPINISE TDLSLLTATV
      1970 1980 1990 2000
VPPSGREEPC LLKRLRNGHV GISFVPKETG EHLVHVKKNG
    2010 2020 2030 2040
QHVASSPIPV VISQSEIGDA SRVRVSGQGL HEGHTFEPAE
            2050 2060 2070 2080
FIIDTRDAGY GGLSLSIEGP SKVDINTEDL EDGTCRVTYC
          2090 2100 2110 2120
PTEPGNYIIN IKFADQHVPG SPFSVKVTGE GRVKESITRR
        2130 2140 2150 2160
RRAPSVANVG SHCDLSLKIP EISIQDMTAQ VTSPSGKTHE
    2170 2180 2190 2200
AEIVEGENHT YCIRFVPAEM GTHTVSVKYK GQHVPGSPFQ
    2210 2220 2230 2240
FTVGPLGEGG AHKVRAGGPG LERAEAGVPA EFSIWTREAG
            2250 2260 2270 2280
AGGLAIAVEG PSKAEISFED RKDGSCGVAY VVQEPGDYEV
          2290 2300 2310 2320
SVKFNEEHIP DSPFVVPVAS PSGDARRLTV SSLQESGLKV
        2330 2340 2350 2360
NQPASFAVSL NGAKGAIDAK VHSPSGALEE CYVTEIDQDK
      2370 2380 2390 2400
YAVRFIPREN GVYLIDVKFN GTHIPGSPFK IRVGEPGHGG
    2410 2420 2430 2440
DPGLVSAYGA GLEGGVTGNP AEFVVNTSNA GAGALSVTID
            2450 2460 2470 2480
GPSKVKMDCQ ECPEGYRVTY TPMAPGSYLI SIKYGGPYHI
          2490 2500 2510 2520
GGSPFKAKVT GPRLVSNHSL HETSSVFVDS LTKATCAPQH
        2530 2540 2550 2560
GAPGPGPADA SKVVAKGLGL SKAYVGQKSS FTVDCSKAGN
      2570 2580 2590 2600
NMLLVGVHGP RTPCEEILVK HVGSRLYSVS YLLKDKGEYT
    2610
LVVKWGDEHI PGSPYRVVVP
```

As used herein, the terms “filamin A modulator” or “FLNA modulator” refer to agents that impact the action of FLNA, by way of non-limiting example by stabilizing one conformation of FLNA at the expense of another, altering the structure of FLNA, altering the expression of FLNA polynucleotide or polypeptide. In various embodiments, the FLNA modulator is an inhibitory nucleic acid. In various embodiments, the FLNA modulator is a small hairpin RNA (shRNA). In various embodiments, the FLNA modulator is a small molecule that binds and reverses an altered conformation of filamin A. In various embodiments, the FLNA modulator is PTI-125.

As used herein, the term “epilepsy” refers to a condition in which a person has recurrent seizures. A seizure is defined as an abnormal, disorderly discharging of the brain's nerve cells (i.e. neurons), resulting in a temporary disturbance of motor, sensory, or mental function.

As used herein, the term “focal cortical dysplasia type II” or “FCD type II” means a disorder of brain development that leads to focal (or discrete) malformations of the cortex with specific cytoarchitecural alterations including (but not limited to) mislamination and neuron dysmorphogenesis. FCD type II can also refer to the malformation itself.

As used herein, “kartogenin” refers to a compound having formula 1:

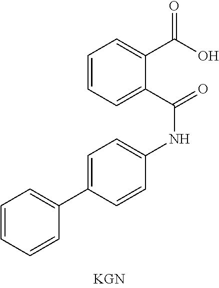 [SMILES: O=C(O)c1ccccc1C(=O)Nc1ccc(-c2ccccc2)cc1]

or a salt, solvate or polymorph thereof.

As used herein, the term “tuberous sclerosis complex” or “TSC” means a genetic disorder resulting from mutations in the gene TSC1 or TSC2 and leads to a spectrum of peripheral and neurological alterations, including, focal malformations of the cortex that are called cortical tubers.

The terms “patient,” “subject,” “individual,” and the like are used interchangeably herein, and refer to any animal, or cells thereof whether in vitro or in situ. amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein “PTI-125” refers to the compound tested in ClinicalTrials.gov Identifier: NCT03748706, hereby incorporated by reference, or salts or solvates thereof. Further details regarding PTI-125 are available in Wang et al., PTI-125 binds and reverses an altered conformation of filamin A to reduce Alzheimer's disease pathogenesis. Neurobiology of Aging. Volume 55, July 2017, Pages 99-114.

As used herein, “treating a disease or disorder” means reducing the frequency or the severity with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e, causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

### Methods of Treating Disease

Without wishing to be limited by theory, the invention is based in part on the discovery that inhibition or modulation of FLNA treats epilepsy associated with hyperactivity of with hyperactivity of the AKT-mTOR signaling pathway and MAPK pathway and the resultant formation of focal cortical malformations (called FCM above) by reducing or preventing seizures associated with these disorders. Accordingly, in one aspect the invention provides a method of treating seizures and thus epilepsy in a subject in need thereof, the method comprising providing to the subject an effective amount of a FLNA modulator. As shown in the examples and figures herein, shRNA mediated knockdown of FLNA and FLNA modulation by PTI-125 have been shown to reduce seizures in animal models.

The use of any FLNA modulator known in the art can be used effectively in various embodiments of the invention. Various FLNA modulators are described in U.S. Pat. Nos. 9,354,223, 8,580,809, 8,614,324 and 9,340,558 as well as U.S. publication No. 2014/028755 and PCT Publication No. WO 2014/028755. Accordingly, in various embodiments, the FLNA modulator is an inhibitory nucleic acid. In various embodiments, the FLNA modulator is PTI-125. In various other embodiments, the FLNA modulator is kartogenin.

In various embodiments the epilepsy is intractable epilepsy. As this term is used herein, intractable epilepsy refers to epilepsy which does not respond or does not satisfactorily respond to other drugs or methods of treatment or whose treatments are not appropriate. In various embodiments, the epilepsy is associated with focal cortical dysplasia (FCD) type II or tuberous sclerosis complex (TSC). FCD type 2 and TSC are conditions known to generate mTORC1 and MAPK (also called ERK1/2) hyperactivity during neurodevelopment and therefore result in the type of focal cortical malformations that FLNA inhibition is shown herein to treat. Accordingly, in various embodiments, the subject has FCD type 2 or TSC. A skilled person will recognize that the invention further provides methods of treating FCD type 2 or TSC by providing a subject in need thereof an effective amount of an FLNA modulator. In various embodiments, the FCDII is due to mutation upstream of mTORC1 but not mTORC1 itself. For example, rheb or TSC mutations lead to increases in both mTORC1 and MAPK activity and MAPK is responsible for FLNA increases

In various embodiments the FLNA modulator is formulated in a pharmaceutical composition comprising at least one pharmaceutically acceptable excipient. The FLNA modulator can be administered by any method of administration known in the art. In various embodiments, the subject is a mammal. In various embodiments, the subject is a human. In various embodiments, the subject is a neonate, juvenile or adult.

In various embodiments, the effective amount of the FLNA modulator is about 0.001-5000 mg/kg, about 0.01-4000 mg/kg, about 0.1-3000 mg/kg, about 0.1-2000 mg/kg, or about 1-1000 mg/kg. In various embodiments, the effective amounts recited immediately above are effective amounts of PTI-125. In various embodiments, the effective amount of PTI-125 is about 0.1-5 mg/kg, about 0.3-3 mg/kg or about 0.5-2 mg/kg. These amounts refer to PTI-125 free base but a person of skill in the art will be readily able to adjust the dose for use with pharmaceutically acceptable salts or solvates of PTI-125. As illustrated in in detail below, in various embodiments the effective amount of the FLNA modulator is about 1-20 mg/kg. In various embodiments the effective amount is about 6-12 mg/kg of PTI-125 salt or about 4-8 mg/kg of PTI free base or equivalent.

In another aspect, the invention provides a method of treating focal cortical dysplasia (FCD) type II or tuberous sclerosis complex (TSC) in a subject in need thereof, the method comprising providing to the subject an effective amount of PTI-125.

In another aspect, the invention provides a method of treating focal cortical dysplasia (FCD), or tuberous sclerosis complex (TSC), or hemimegalencephaly in a subject in need thereof, the method comprising providing to the subject an effective amount of kartogenin.

### Methods of Treating and Detecting Neurodegenerative Disease

FLNA modulators are recognized as inhibiting hyperphosphorylation of tau protein and therefore may be useful for the treatment of neurodegenerative diseases including but not limited to Alzheimer's disease and Parkinson's disease. Accordingly, in another aspect, the invention provides a method of inhibiting hyperphosphorylation of the tau protein that comprises the steps of administering to cells of the central nervous system in recognized need a FLNA-binding effective amount of kartogenin or a pharmaceutically acceptable salt.

FLNA modulators have been shown to alter the conformation of FLNA and shift the equilibrium of certain protein-protein complexes that are involved in Alzheimer's disease pathology in a manner that can be employed to detect Alzheimer's disease if present in a subject. Accordingly, in another aspect the invention provides a method for determining the likelihood of a living patient having Alzheimer's disease pathology (AD pathology) comprising the steps of determining the amount of one or more of a protein-protein complex i) α7nAChR/FLNA, ii) TLR4/FLNA and iii) α7nAChR/Aβ present in a first portion of a lymphocyte preparation from said living patient: determining the amount of said one or more of i) α7nAChR/FLNA, ii) TLR4/FLNA and iii) α7nAChR/Aβ present as a protein-protein complex in a second portion of said lymphocyte preparation, said second portion of said lymphocyte portion further containing admixed therein a FLNA binding-effective amount of kartogenin or a pharmaceutically acceptable salt thereof; comparing the values so determined. whereby a determined amount of said one or more of i) α7nAChR/FLNA, ii) TLR4/FLNA and iii) α7nAChR/Aβ present as a protein-protein complex in a second portion of said lymphocyte preparation that is significantly decreased in the presence of kartogenin or pharmaceutically acceptable salt thereof indicates that the patient had AD pathology at the time the body sample was taken, whereas no significant difference between the two determined values indicates that the patient was free of AD pathology at the time the body sample was taken.

### Administration/Dosage/Formulations

The regimen of administration may affect what constitutes an effective amount. The therapeutic formulations may be administered to the subject either prior to or after the onset of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease in the patient. An effective amount of the therapeutic compound necessary to achieve a therapeutic effect may vary according to factors such as the state of the disease or disorder in the patient: the age, sex, and weight of the patient; and the ability of the therapeutic compound to treat a disease in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40) mg to about 900 mg, about 50 mg to about 800 mg, about 60 mg to about 750 mg, about 70) mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose: granulating and disintegrating agents such as cornstarch: binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of the disease in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the invention is optionally given continuously: alternatively, the dose of drug being administered is temporarily reduced or temporarily suspended for a certain length of time (i.e., a “drug holiday”). The length of the drug holiday optionally varies between 2 days and I year, including by way of example only, 2 days, 3 days, 4 days, 5 days, 6 days, 7 days, 10 days, 12 days. 15 days, 20 days, 28 days, 35 days, 50 days, 70 days, 100 days, 120 days. 150 days, 180 days, 200 days, 250 days, 280 days, 300 days, 320 days, 350 days, or 365 days. The dose reduction during a drug holiday includes from 10%-100%, including, by way of example only. 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Study Design

The objectives herein were to investigate whether the expression of FLNA was increased in tissue from FCDII individuals and the cortices of mice modeling FCDII, and to then examine whether decreasing the amount of FLNA or its function was sufficient to reduce seizures. To decrease FLNA expression, an shRNA strategy was used as previously reported. The shRNA plasmid and FLNA antibody were validated. The quantity of shRNA to used was based on the previous study. To block FLNA function, a small molecule PTI-125 was used. The dosage of PTI-125 was determined by first testing the effects of PTI-125 on cell placement and morphology. The treatment paradigm was chosen to match several developmental milestones. Experimental and control animals were littermate although several litters had to be used for most experiment, and none were excluded from analysis at the time of harvest. Experimental animals were lost prior to or during seizure recordings either due to sudden death by seizure or due to technical issues with the recording system. No attempt was made to segregate results based on gender or sex. Analysis was performed blindly and seizure analysis was performed by two investigators. In addition, mice were randomly split into different groups for drug injection. Blinding was performed at multiple points during the experiments. For example, after video-EEG recording, the recording files were renamed for analysis by investigators blinded to the treatment condition.

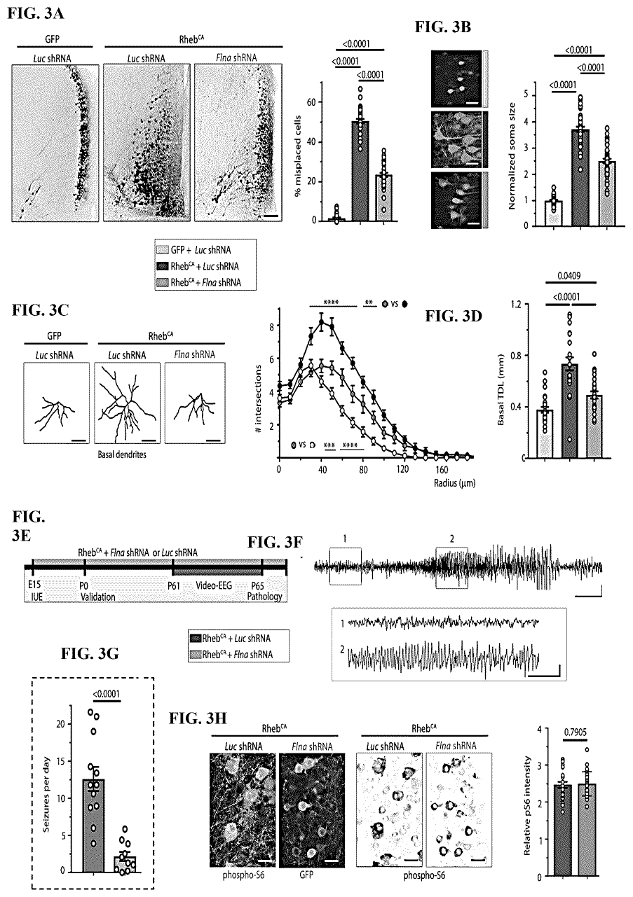

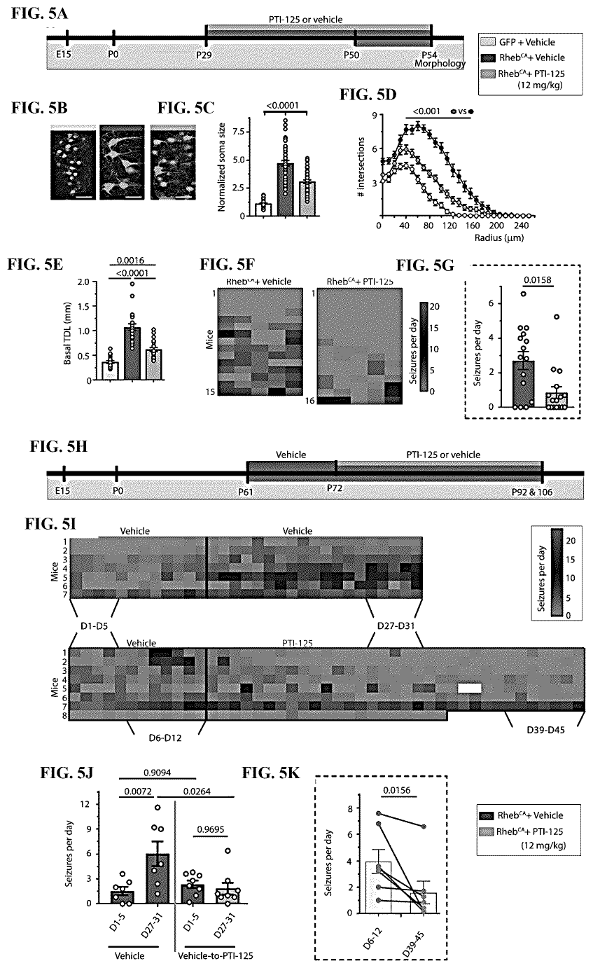

Normality was checked with D'Agostino And Pearson normality test. Sample sizes were estimated based on our previous experience both for studying the anatomy and seizure activity. The sample size calculation was performed using power analysis with G*Power 3. For each set of experiment, the sample size was estimated for an effect size of 50% using SD calculated from the control population and a power at 80% (B=0.2) and an a of 0.05. Post-experiment, for FIG. 5G for example (effect of PTI-125 treatment from P29 to P54 on seizure frequency), using a Mann-Whitney test (2 groups, non-equal SD and not normal distribution, two tails), 13 mice in each group would be sufficient to reach significance with a power of 0.9 and an a of 0.05 for the presented data. Each experiment was reproduced at least three times with n greater or equal to three as detailed in Table 3. One mouse was removed for the video-EEG data in FIG. 3G. The mouse displayed no seizure despite bilateral electroporation of Rheb^{CA} and after examination of the brain sections, very few electroporated cells were found per section (<10 cells). Electroporation was thus unsuccessful. The brain of every recorded mouse was examined post-EEG recording to make sure that the mouse with no seizure had proper electroporation.

### Animals

All experiments were performed on CD-1 mice (Charles River) of either sex.

### Human Tissue Sample

Human tissue was obtained directly from the surgery room and rapidly frozen in liquid nitrogen and then stored at −80° C. (for immunoblotting and immunostaining) or fixed in 10% formalin (for immunohistochemistry). Patient information is provided in Tables land 2. The mean age of the control patients and patients with FCDII were 13.5±2.02 (n=11) and 15.08±2.47 years (n=12), respectively.

### In Utero Electroporation and Plasmids

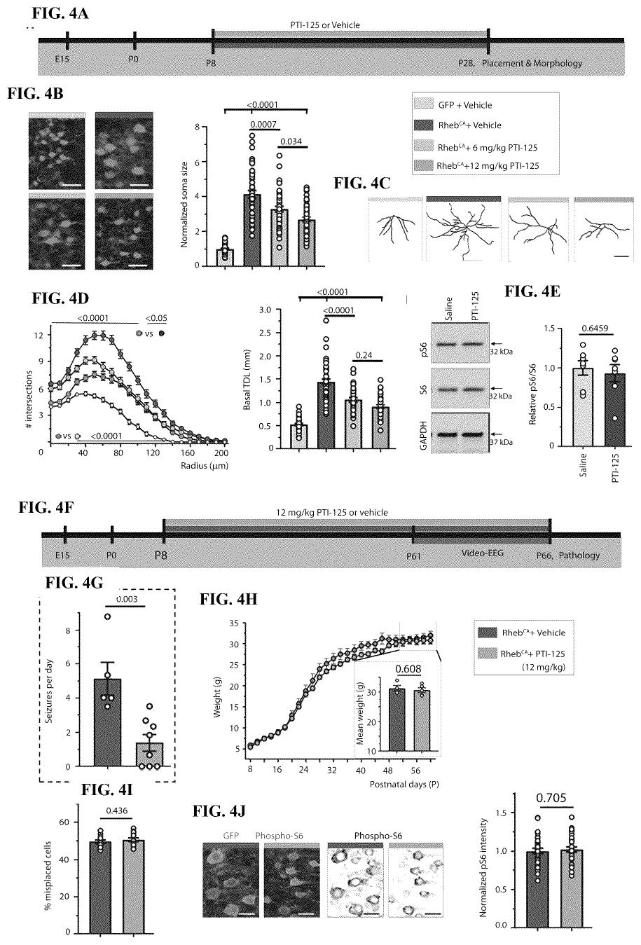

Each DNA plasmid was diluted in sterile PBS (pH 7.4). Timed pregnant mice (embryonic day, E15) were anesthetized with isoflurane. After exposing the uterine horns, ˜1.5 μl of DNA solution, containing 0.1% fast green added as an injection tracer, was injected into the lateral ventricle via a pulled glass capillary. PBS-soaked tweezer-type electrodes (model 520, BTX) were then positioned on the head of the fetuses across the uterine wall and 6 square-pulses at 42 mV and 50 ms duration every 950 ms were applied using a pulse generator (ECM830, BTX). At birth, mice were prescreened for successful electroporation by detecting the expression of fluorescent protein markers on a fluorescence-enabled stereo microscope (SZX16, Olympus). Morphology and seizure data in different conditions (Flna vs Luciferase shRNA or vehicle vs PTI-125 treatment) were compared between littermate mice. For most experiments, mice received unilateral electroporation, except for experiments in FIG. 4F (P8-P65 PTI-125 treatment on seizures) for which mice received bilateral electroporation.

### Transcardial Perfusion and Immunofluorescence

Mice: Mice were transcardially perfused with PBS followed by 4% paraformaldehyde. Brains were then dissected and post-fixed in 4% paraformaldehyde for 24 hours before being transferred to a solution of 3% agarose solution in PBS. These were then sectioned coronally on a vibratome at a thickness of 50 μm. For immunofluorescence, free-floating sections were incubated for 1 hour in a blocking solution (2% bovine serum albumin and 0.1% Triton-X in PBS) at room temperature and then incubated with primary antibodies (Table 5) overnight at 4° C. Sections were washed with PBS+0.05% Tween 20 and then placed in blocking solution containing secondary antibodies for 1 hour at room temperature. All images were taken using an FV1000 confocal microscope (Olympus).

Humans: For Immunohistochemistry analysis, human samples were frozen in liquid nitrogen or fixed in 10% formalin and embedded in paraffin for preparing serial sections (4 μm-thick) that were then deparaffinized and rehydrated by immersion in xylene followed by ethanol gradients. Slices were soaked in citrate solution followed by boiling in a microwave (for antigen unmasking) and washed in 3% hydrogen peroxide. Tissue sections were incubated with primary antibodies (Table 5) overnight at 4° C., in a moist chamber. After washing with PBS, slices were incubated with secondary antibody for 30 mins at room temperature. Sections were mounted after dehydration.

Quantification of Neuronal Distribution. Soma Size. Staining Intensity.

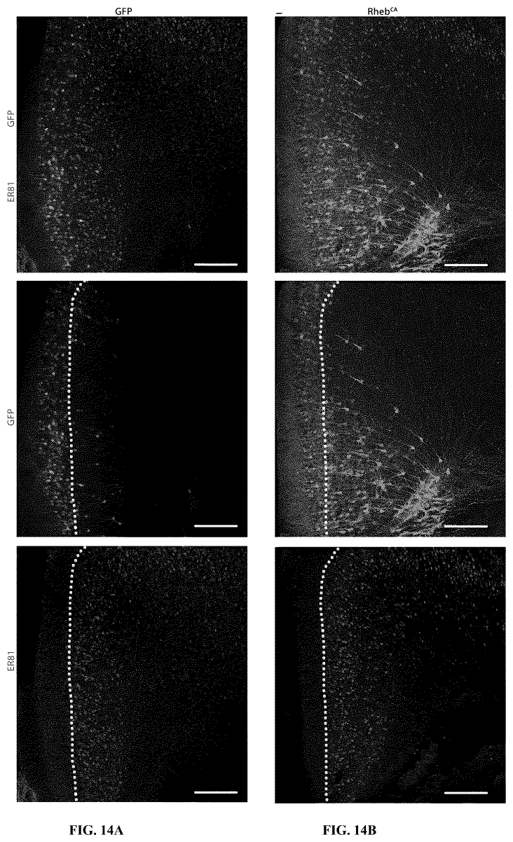

Quantification of the number of layer 2/3 neurons in the anterior cingulate cortex, the boundary between layer 2/3 and layer 5 was determined using ER81 staining, which labels layer 5 (FIGS. 14A-14B) Soma size was quantified by outlining the soma of GFP+ cells and measuring the area using ImageJ. Intensity of 27pproxim-S6 and FLNA in sections was quantified by outlining somas of cells and measuring the mean gray value. Then, the values in electroporated cells (in mice) or SMI-311-positive cells (in humans) were normalized to those in non-electroporated cells or unstained cells surrounding electroporated cells to control for variability of staining between sections.

### Sparse Neuron Electroporation and Quantification of Dendritic Trees

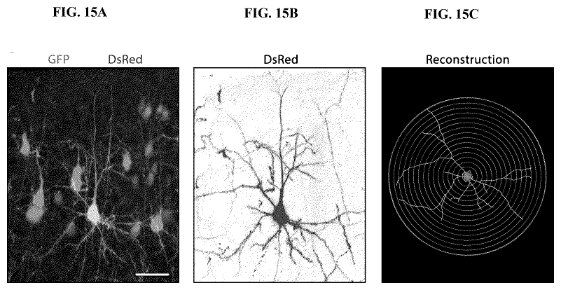

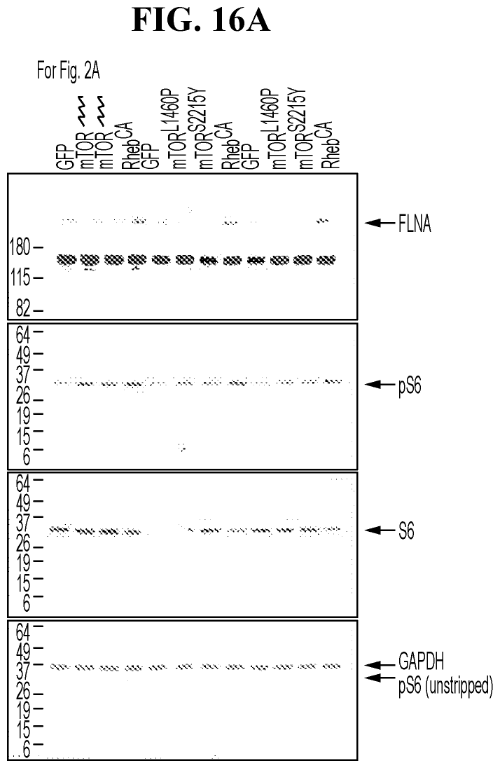

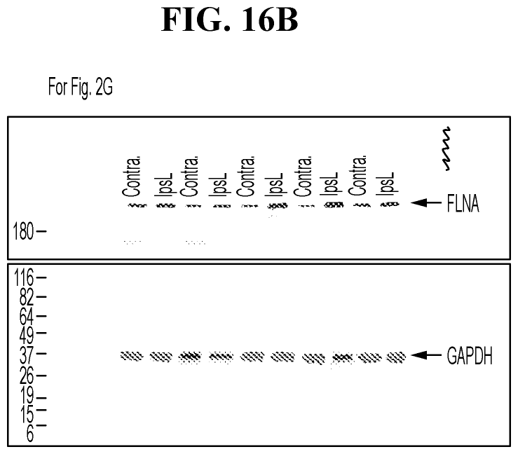

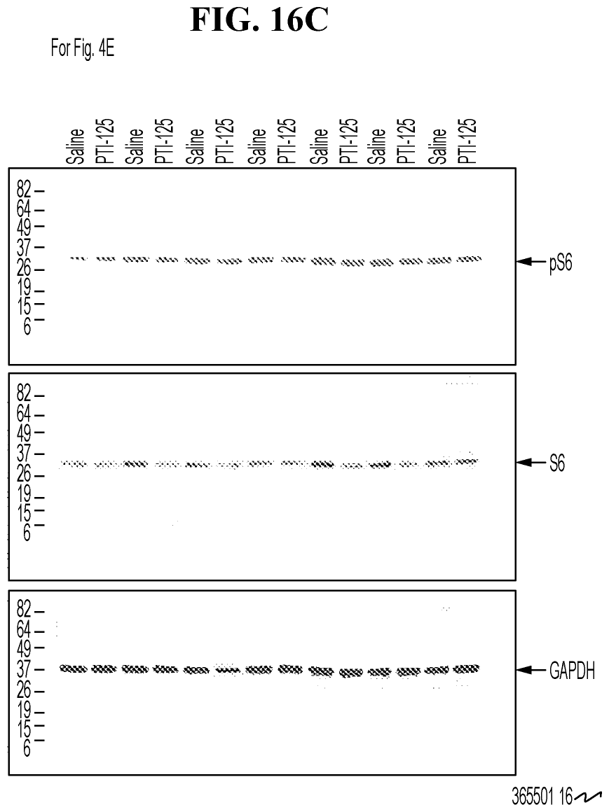

To analyze neuronal morphology and, in particular dendritic trees, only a few neurons per section were labeled using a Cre-based strategy as previously reported. More specifically, an inducible DsRed2 vector (pCAG-LoxP-stop-LoxP-DsRed2, pCALNL-DsRed2) combined with a Cre vector (pCAG-Cre) was used at very low concentration (1 ng/μl). The Cre plasmid led to DsRed expression in only 3-5 neurons per slice allowing to cleanly analyze the dendritic morphology of individual neurons (FIGS. 15A-15C) Images of DsRed-expressing basal dendrites were acquired in coronal sections using a Fluoview 1000 confocal microscope with a 20× objective. Basal dendrites were traced with simple neurite tracer software (FIJI and Neuronstudio). Sholl analyses were carried out using the number of intersections in 10-μm-increment concentric circles as a measure of morphological complexity. Z stacks from three different square fields of view were taken from three different sections. Analysis was performed blindly with more than four animals and more than 20 cells per condition (see Table 3 for actual n number).

### Neuro2a Cell Culture and Transfection

Neuro2a cells were grown at 37° C., with 5% CO₂ in a complete media consisting of high glucose DMEM (Gibco, #11965-092), 5% heat-inactivated fetal bovine serum (Gibco, #16140071), and 1% penicillin-streptomycin. Cells were plated in 12 well culture plates (Corning, #0720082) and transfected when they reached ˜70% confluence. Transfection was done using Poly Jet transfection reagent (Signagen, SL100688) according to the manufacturer's instructions. Cells were lysed 72 hours after transfection for all experiments.

### Nucleofection and Culture of E15 Cortical Neurons

The medial prefrontal cortexes of E15 pups were dissected out and incubated in papain digestion solution (Worthington, #LK003176) for 15 min at 37° C. The cortexes were then transferred to plating medium (MEM supplemented with 5% FBS, 0.45% glucose) and dissociated by using glass pipette. Following dissociation, cells were nucleofected using a Mouse Neuron Nucleofector kit (Lonza, #VPG-1001) according to the manufacturer's instructions. Cells were finally plated on poly-D-lysine-coated 6-well plates (BD Biosciences, #354413). The medium was changed to neuronal maintenance medium (neurobasal with 1×B27 and 1× GlutaMAX-1) 2 h after plating. Protein extraction and western blots were performed 14 days after nucleofection.

### Western Blot

Human and mouse samples were lysed in RIPA buffer (Thermo fisher, #89900) with protease/phosphatase inhibitor cocktail (Cell Signaling Technology, #5872), 5 mM EDTA, and 20 units/ml Dnase I (Roche). All lysates were run on Tris-glycine gels (Bio-Rad, #456-1086 for mouse samples and #456-1046 for human samples). Proteins were transferred to PVDF and blocked in 5% milk and incubated with primary antibodies (concentrations of primary antibody are listed in Table 5). HRP-conjugated anti-rabbit or anti-mouse were used as secondary antibodies. Phosphorylated and total proteins were blotted on the same membrane after stripping (Thermo fisher, #21059). All density measurements were performed using NIH Image J software. In the case where cross-blot normalization was required a reference sample was loaded on each gel to account for inter-gel variability (FIGS. 11A-11B).

### EEG Headmount Implantation

Prefabricated EEG headmounts (Pinnacle Technology, cat. No. 8201-EEG) were implanted in 6 to 8 weeks old mice. Mice were anesthetized with isoflurane and positioned on a stereotaxic frame using ear bars. A rostro-caudal midline incision was made in the skin to expose the skull surface. Four pilot holes (two bilateral holes 1 mm anterior to bregma and two bilateral holes 5 mm posterior to bregma, each 1.5 mm lateral to sagittal suture), was tapped through the skull to dura using a microdrill (Roboz Surgical Instrument, #RS6300). The headmount was attached on top of the skull by threading four stainless steel screws (Pinnacle Technology, cat. No. 8209) into the pilot holes. Silver conductive paint (Electron Microscopy Science) was applied around the screw threads to ensure a solid connection with the headmount. The entire implant was insulated using dental acrylic. Mice were allowed to recover in their home cage for seven days before video-EEG monitoring.

### Video-EEG Recordings and Analysis

Mice were housed in individual recording chambers in a light-, temperature- and humidity-controlled room during video-EEG monitoring. Mice were freely moving inside the chambers and had ad libitum access to food and water. Synchronous video-EEG recording was acquired using a three-channel EEG tethered system (Pinnacle Technology, cat. No. 8200-K1-iSE3) and Sirenia Acquisition software (Pinnacle Technology). Mice were continuously recorded 24 hours/day for 5-6 consecutive days or 31-45 days.

Seizure frequency and duration were analyzed using Sirenia Seizure Basic software (Pinnacle Technology). All analyses were performed blinded to experimental groups by at least two investigators. The entire EEG traces were manually reviewed for the occurrence of seizures, defined as a sudden onset of high amplitude activity with a characteristic pattern of progressive frequency and amplitude changes over the course of the event lasting ≥10 sec. Seizure onset was defined by high amplitude firing >2× baseline and seizure termination was defined by a return to baseline activity. Video data were inspected for behavioral correlates including myoclonic jerks, tonic-clonic activities, convulsions, and loss of postural control (rearing and falling) and were used as secondary verification of seizures. For each individual animal, mean number of seizures per day was obtained by dividing the total number of seizures by the total number of recording hours and multiplied by 24.

### Drug Injections

PTI-125 was dissolved in saline at a concentration of 2.4 mg/ml. Mice received two intraperitoneal injections of 6 or 12 mg/kg PTI-125 every day. Saline was used as vehicle treatment.

### Statistical Analyses

All analyses were conducted blindly knowing only the arbitrarily assigned animal ID (independent of electroporation condition). Statistical tests and plots were performed using Prism 7 (GraphPad Software, Inc.). Statistical significance was determined using Student's t-test (two-tailed, paired or unpaired), one-way ANOVA, two-way ANOVA (repeated measures, with Sidak or Tukey multiple comparisons post-test), Wilcoxon matched-pairs signed rank test. Mann Whitney U test (two-tailed), and Fisher Exact test, with P<0.05 for significance for all experiments. Data are presented as mean±SEM. Table 3 details the statistical test used for each data set as well as the n.

### Example 1: FLNA is Increased in Patients with FCDII and in Rheb^{CA} Mice Modeling TSC and FCDII

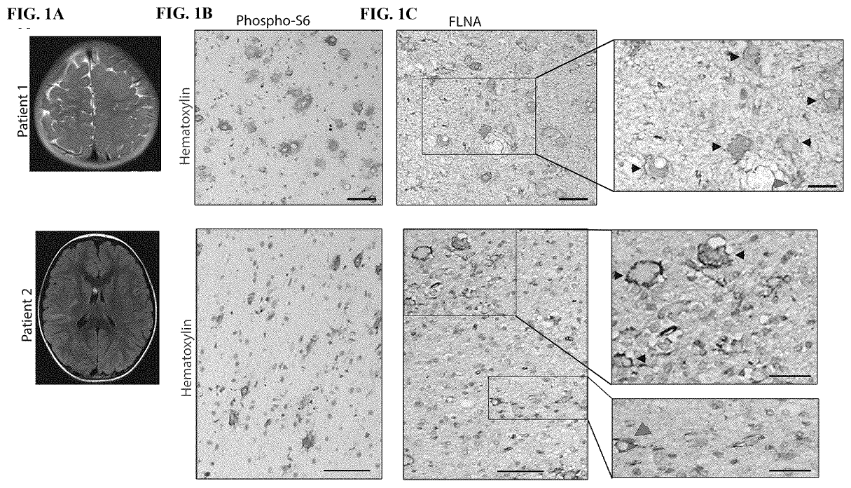

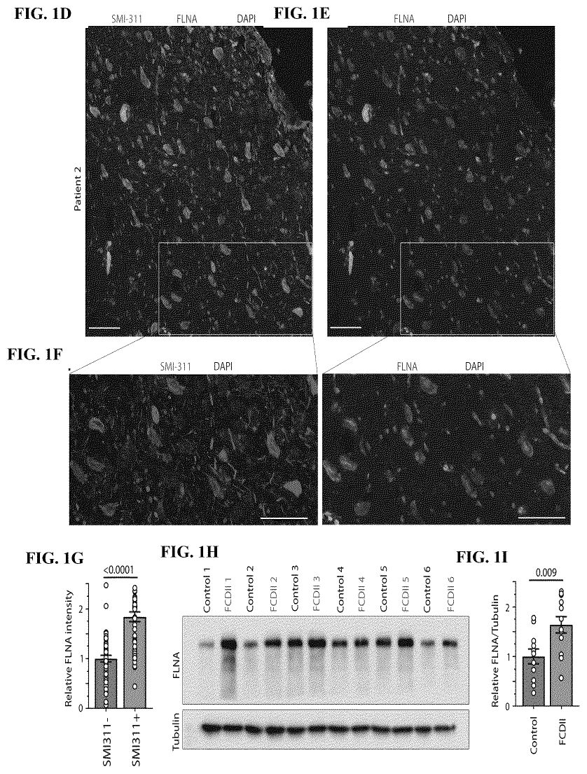

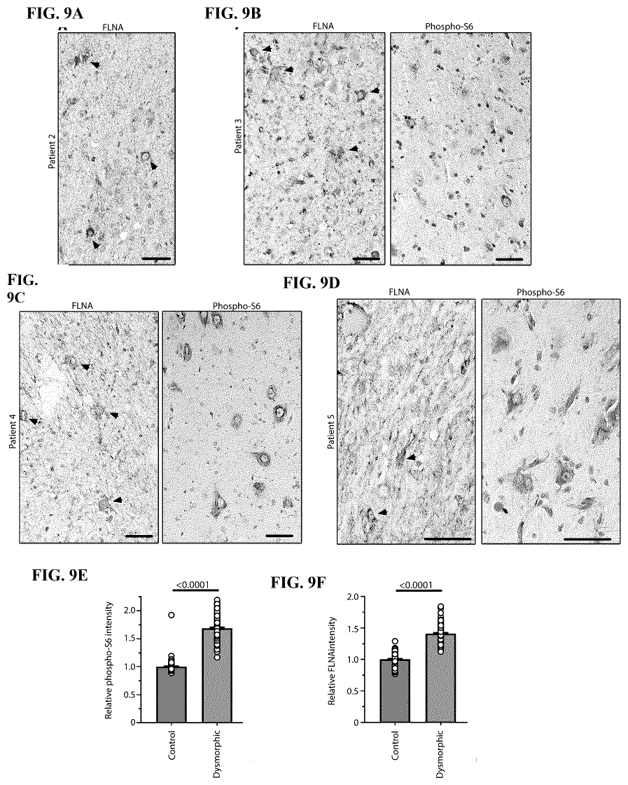

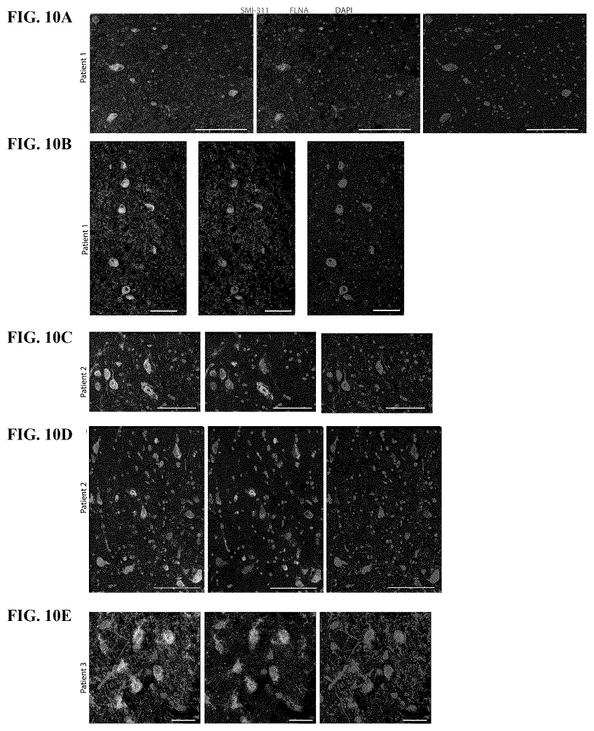

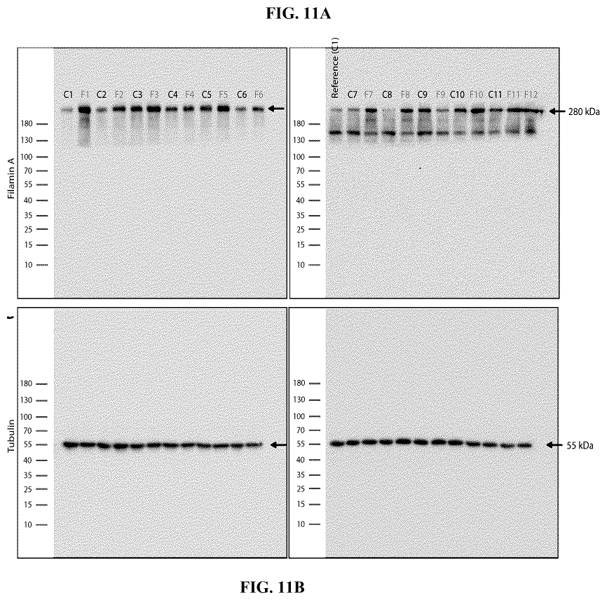

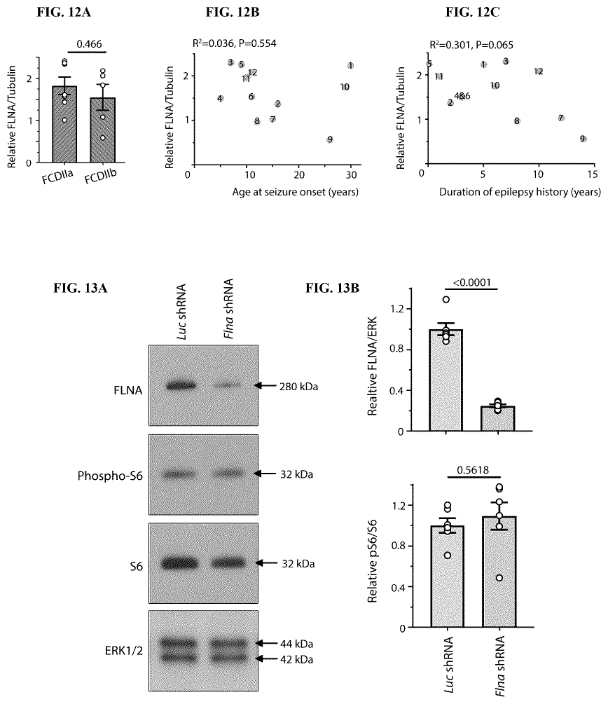

Cortical tissue samples from 17 patients with FCDII who underwent surgery for epilepsy were obtained (Table 1). Every patient had FCM detected on MRI scans (two examples are shown in FIG. 1A) and underwent electroencephalogram (EEG) recording with a combination of subdural grid and depth electrodes prior to FCM resection. All patients were identified as FCDII post-surgery based on pathological examination of the hematoxylin-stained resected tissue and identification of hallmarks of FCDII, including cortical dyslamination and the presence of cytomegalic, dysmorphic neurons (Table 1). The distinction between FCDIIa and FCDIIb was based on the absence or presence of balloons cells, respectively. For the FCDII tissue examined by immunohistochemistry (n5), the presence of dysmorphic cells was confirmed, including classical multinucleated balloon cells, and an increase in phosphorylated ribosomal protein S6 (30pproxim-S6), a read-out of mTORC1 activity (FIG. 1B, and FIGS. 9A-9F). These enlarged cells had increased expression of FLNA compared to surrounding normal-sized cells (FIG. 1C and FIGS. 9A-9F). To verify that cells expressing FLNA were neurons. FLNA and SMI-311 were co-immunostained for, a marker of cytomegalic, dysmorphic neurons. All SMI-311-positive neurons were immunoreactive for FLNA (FIGS. 1D-1F, and FIGS. 10A-10E) and displayed significantly increased FLNA intensity compared to surrounding cells (P<0.0001, FIG. 1G).

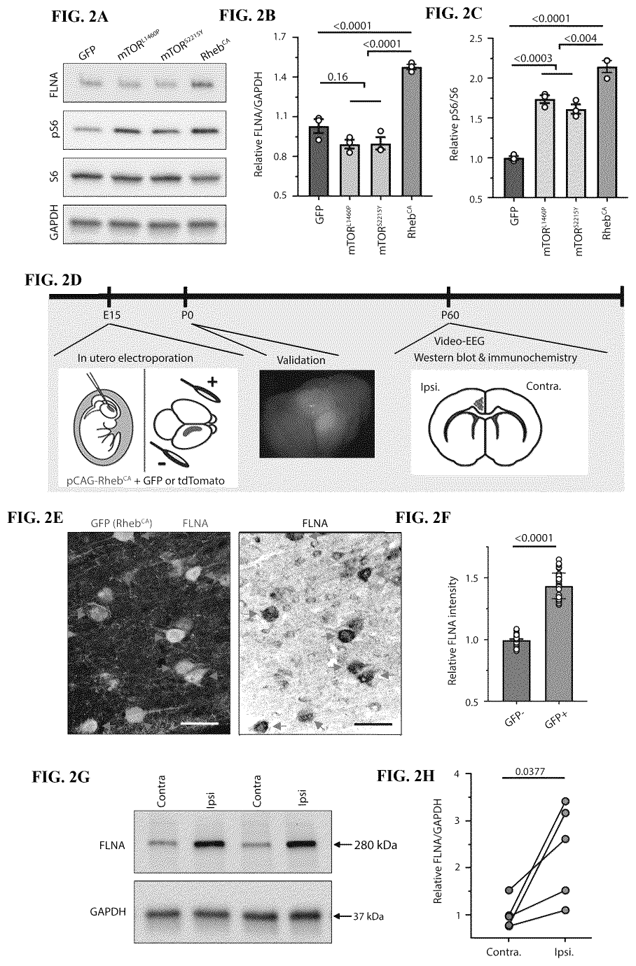

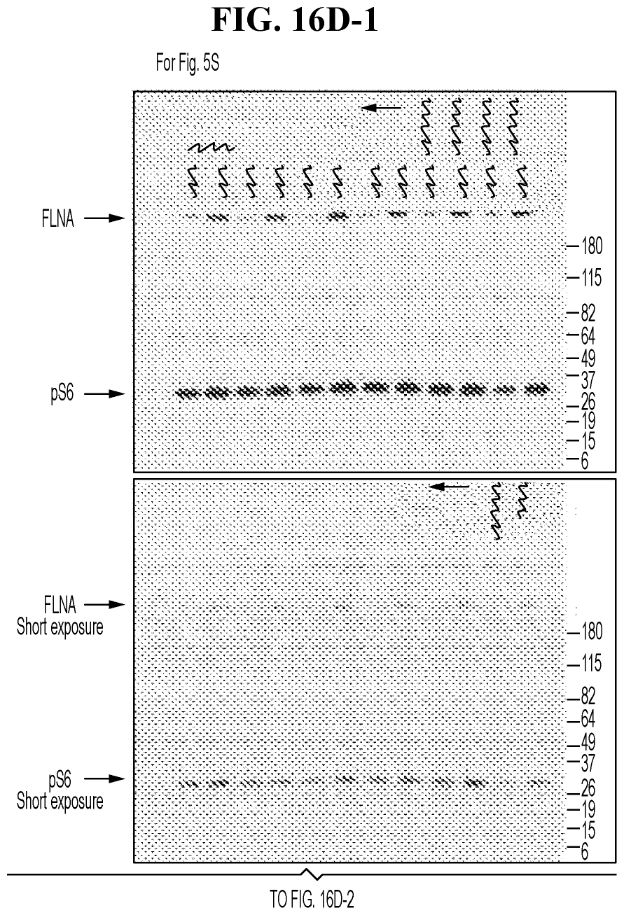

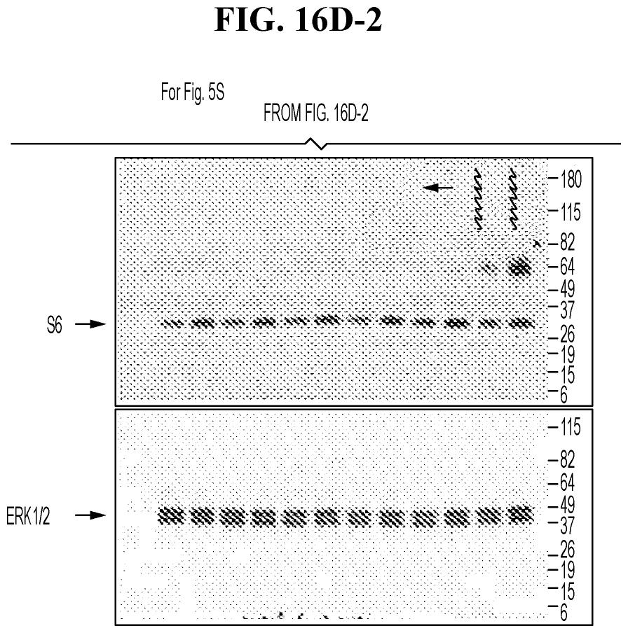

To quantify FLNA expression in patients with FCDII (n=12) compared to controls, tissue from age-matched control patients who underwent surgery for brain trauma (n=11. Table 2) were obtained and immunoblotting for FLNA was performed (FIG. 1H). As a group. FCDII tissue from 12 patients displayed significantly (P=0.009) increased amount of FLNA compared to control cortical tissue (FIG. 11 and FIGS. 11A-11B: numbers on top of the blot correspond to patient numbers in Table 1). In light of the variability of the western blot data (ranging from 0.57 to 2.31× control values), it was examined whether the amount of FLNA was correlated with the age of seizure onset, the duration of epilepsy history, and the diagnosis of FCDIIa versus FCDIIb. No correlation was found between the amount of FLNA and these parameters (FIGS. 12A-12C). Another parameter that may contribute to the variability in FLNA expression is the identity of the mutant genes. Indeed, as previously reported the increase in FLNA induced by Tsc1 loss or Rheb^{CA} overexpression was mTORC1-independent, suggesting that mutations in mTORC1 itself do not contribute to increases in FLNA. This hypothesis was tested in cultured neurons (14 days in vitro) nucleofected with two plasmids encoding mTORC1 with gain-of-function mutations reported in patients with FCDII or Rheb^{CA}. The two mTORC1 mutations did not increase FLNA compared to control whereas Rheb^{CA} did, despite that all three conditions significantly increased 31pproxim-S6 (P<0.0003, FIGS. 2A-2C and FIGS. 16A-16D-2). Although the mutations in the human tissue used for immunoblotting were not identified, these data may explain some of the variability in the amount of FLNA in human samples. These data suggest that FLNA could be increased in patients with TSC or FCDII resulting from PI3K-Rheb pathway gene mutations but not from mTORC1 mutations.

Prior to examining the contribution of increased FLNA to seizures, it was examined whether FLNA was increased in our Rheb^{CA} mouse model of TSC and FCDII-like FCM associated with seizures. This model was generated using in utero electroporation (IUE) of plasmids encoding Rheb^{CA} and the fluorescent reporter green fluorescent protein (GFP) in the medial prefrontal cortex at embryonic day I 15 (FIG. 2D). In control, littermate mice, tdTomato was expressed instead of Rheb^{CA}. The cortex of Rheb^{CA}-expressing mice displayed the cytoarchitectural hallmarks of human FCM, including cell misplacement, increased soma size, and neuronal dysmorphogenesis (FIGS. 3A-3D). The animals exhibited daily. convulsive seizures as monitored by video-EEG at 2-3 months of age. FLNA immunostaining in cortical sections of Rheb^{CA}-expressing mice illustrated increased FLNA in Rheb^{CA}-expressing cells (GFP+) compared to surrounding, non-electroporated (GFP−) cells (FIGS. 2E and 2F). In addition, immunoblots confirmed increased FLNA in the FCM-containing cortices (ipsilateral) compared to the contralateral (non-electroporated) cortices (FIGS. 2G and 2H, and FIGS. 16A-16D-2).

### Example 2: Normalizing FLNA Expression in Dysmorphic Neurons of mTORC1-Driven FCM Attenuates Seizure Frequency

It was examined whether normalizing FLNA expression would prevent some of the cytoarchitectural abnormalities found in the experimental FCM. To decrease FLNA expression in Rheb^{CA}-expressing neurons, a short hairpin RNA (shRNA) against Flna or Luciferase (control) were co-expressed using E15 IUE. A 1:1 ratio of FLNA shRNA to Rheb^{CA} plasmid concentration was used because it was previously reported that this ratio fully normalized FLNA expression in Rheb^{CA}-containing cortical neurons in vitro. Cell placement and morphology were analyzed at postnatal day (P) 28 as previously reported. Flna shRNA partially, but significantly, prevented neuronal misplacement (from 50% to 23% of misplaced cells versus only 2% in control. P<0.0001. FIG. 3A), increased soma size (from 372% to 250% of control. P<0.0001. FIG. 3B), and dendritic dysmorphogenesis (from 194% to 130% of control total dendritic length. P<0.0001. FIGS. 3C and 3D. Considering that increased soma size is often used as a read-out of increased mTORC1activity and that decreasing FLNA expression reduced soma size, it was examined whether Flna shRNA would affect the amount of 32pproxim-S6, a more direct read-out of mTORC1 activity than soma size. Flna shRNA did not prevent increased 32pproxim-S6 intensity in vivo (FIG. 3H). This was further confirmed in vitro in Neuro2a cells in which Flna shRNA did not reduce Rheb^{CA}-induced increased S6 phosphorylation (FIGS. 13A-13B). In addition, although Flna shRNA reduced Rheb^{CA}-induced increase in soma size, it did not normalize it (still increased by 250%) consistent with persistent hyperactive mTORC1 in the Flna shRNA condition. Finally, video-EEG recordings of Rheb^{CA}-expressing mice for 5 days starting at P61 followed by pathological analysis of the tissue (FIGS. 3E and 3F) was obtained. Mice expressing Flna shRNA in Rheb^{CA}-expressing neurons displayed a significantly lower seizure frequency (by 83%. P<0.0001) compared to mice expressing luciferase shRNA (mean of 2.2 vs 12.6 seizures/day. FIG. 3G). In addition, whereas 4 out of 16 mice died in the control group, no mice died in the Flna shRNA group (out of 10 mice). Both groups of mice (Flna and Luciferase shRNA) had similar increases in 32pproxim-S6 expression (FIG. 3H). These data suggest that differences in seizure activity between the control and Flna shRNA condition were not due to variations in Rheb^{CA}-induced mTORC1 activation, and thus FCM features. and that the rescue of cytoarchitectural abnormalities by Flna shRNA was independent of mTORC1.

### Example 3: Long-Term Treatment with a Small Molecule Modulator of FLNA, PTI-125, Prior to Seizure Onset Partially Prevents Neuronal Dysmorphogenesis and Reduces Seizure Activity

The therapeutic potential of modulating aberrant FLNA activity via a small FLNA-binding molecule. PTI-125 was investigated. PTI-125 was derived from an iterative in silico in vitro screening process against a known pentapeptide region of FLNA that was identified in an earlier study. PTI-125 has been reported to bind native FLNA as well as aberrant FLNA in Alzheimer's disease with picomolar and femtomolar affinity, respectively. and is currently in clinical trials for Alzheimer's disease (ClinicalTrials.gov no. NCT04079803). Twice-daily intraperitoneal injections of 6 or 12 mg/kgPTI-125 (salt form) were given to Rheb^{CA}-expressing mice from P8 to P28 to test efficacy at preventing the FCM-associated cytoarchitectural abnormalities (FIG. 4A). P8 was chosen because it corresponds to a new born human and by P28 dendritic development is complete. PTI-125 at 6 and 12 mg/kg partially rescued neuronal soma size and dendritic dysmorphogenesis, with slightly greater efficacy at the 12 mg/kg dose (FIG. 4B-4D). It was also found that 33pproxim-S6 expression in the cortex of nonelectroporated mice were not affected by PTI-125 (12 mg/kg) treatment (FIG. 4E and FIGS. 16A-16D-2). Next was examined the efficacy of long-term (P8-P65) PTIs-125 treatment at 12 mg/kg on seizure activity that was recorded at P61 to P66 using video-EEG (FIG. 4F). Littermate mice were randomly split into two groups, receiving PTI-125 or saline vehicle (intraperitoneal injections twice daily). PTI-125 treatment significantly reduced seizure frequency compared to vehicle treatment (5.1 vs 1.4 seizures/day. P=0.003. FIG. 4G). This treatment did not affect body weight (FIG. 4H). In addition, cell misplacement and 33pproxim-S6 intensity in brain sections from recorded mice were not affected by PTI-125 treatment (FIGS. 4I and 4J). Hence, the reduced seizure activity in PTI-125-treated mice was not due to technical issues with IUE or differences in mTORC1 activation between the two groups of mice. Thus. PTI-125 treatment prior to seizure onset efficiently reduces seizure frequency by 73% independently of mTORC1.

### Example 4: PTI-125 after Seizure Onset Rescues Neuronal Dysmorphogenesis and Decreases Seizure Activity

Next tested was whether a treatment with PTI-125 after the onset of seizures could rescue some of the morphological abnormalities and seizure activity. In our mouse model. Racine grade 4-5 seizures are visible by P21. A 26-day treatment with PTI-125 from P29 to P54 (3-week treatment. FIG. 5A) partially, but significantly, rescued increased soma size (P<0.0001. FIGS. 5B and 5 C) and dendritic dysmorphogenesis (P<0.0001. FIG. 5D and FIG. 5E). To assess efficacy on seizure activity, video-EEG recordings were obtained at the end of the treatment (P50-P54). PTI-125-treated mice displayed a significantly lower seizure frequency by 69% (P=0.0158) compared to vehicle-treated mice (0.84 vs 2.7 seizures/day. FIGS. 5F and 5G). Then assessed was the effect of PTI-125 on seizure activity in young adult (P61-P106) mice that were recorded during saline injections and subsequently switched to PTI-125 injections to assess treatment effects in the same mice (FIG. 5H). Recorded also were randomly selected littermate mice that were treated with vehicle from P61 to P92 and observed that seizure frequency increased over time (FIGS. 5I and 5J). In that same timeframe, corresponding to a 20-day PTI-125 treatment, littermate mice that were switched from vehicle to PTI-125 after 12 days of recordings did not have the increase in seizure frequency observed in the vehicle group (FIGS. 5I and 5J). Moreover, extended recording of the PTI-125-treated mice showed that a 33-day treatment led to a significant 60% reduction (P=0.0156) in seizure frequency (mean of 1.6 vs 3.9 seizures/day, FIG. 5K). Thus, PTI-125 treatment after the onset of seizures in either juvenile or adult mice decreased seizure frequency and prevented the worsening of seizure activity observed over time in vehicle-injected adult mice.

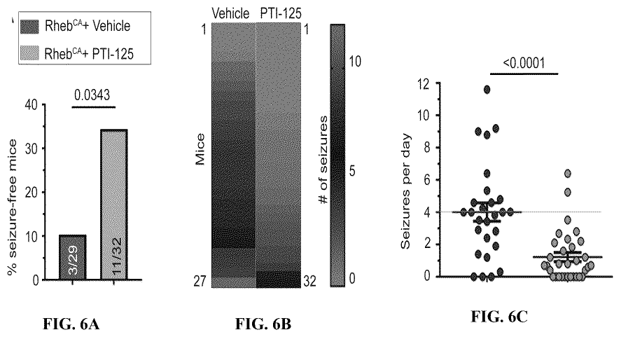

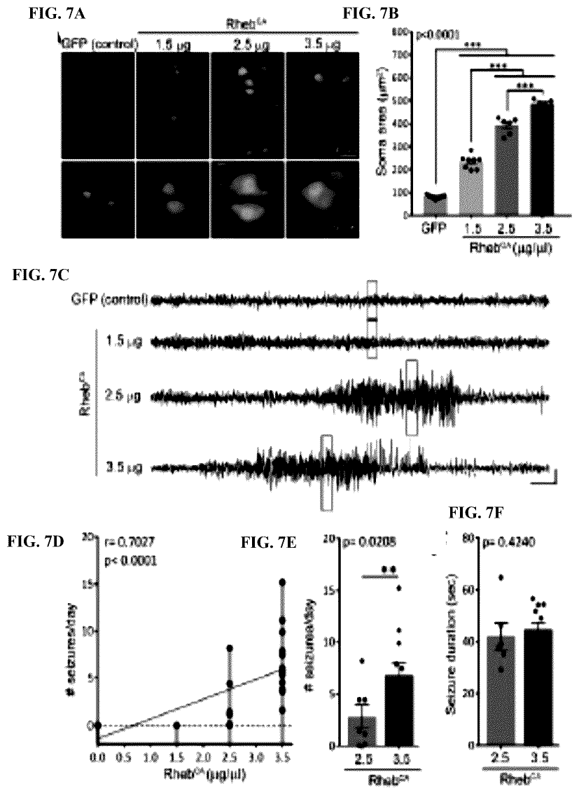

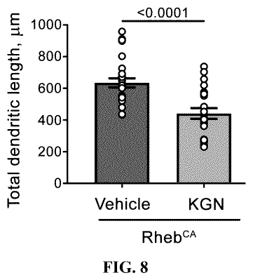

Finally, the seizure data obtained for all the PTI-125 treatments were combined and compared to their corresponding vehicle treatments (data from FIGS. 4G, 5G, and 5K corresponding to P8-P65, P29-P54, and P71-P91 treatments, respectively). PTI-125 treatments significantly increased the number of seizure-free mice (35% vs 11%, P=0.0343, FIG. 6A). In addition, mice treated with PTI-125 (n=32) exhibited an overall 66% reduction in seizure frequency compared to vehicle-treated littermates (n=27), illustrated in a heatmap (FIG. 6B) and scatter graph (P<0.0001, FIG. 6C).

### Example 5

It was found that cortical tissue from patients with FCDII displayed increased FLNA. In addition, the increase in FLNA was observed in SMI-311-immunopositive enlarged neurons and cells resembling balloon cells. These findings are in agreement with study reporting increased FLNA expression in Tsc1^{null} neurons in vivo and Rheb^{CA}-expressing cortical neurons in vitro as well as in cortical tissue from individuals with TSC. However, there was a marked variability in the amount of FLNA in human FCDII samples, including samples that expressed similar FLNA amount to that in controls. This variability could be explained by the fact that mutations leading to increased Rheb activity led to FLNA increases whereas gain-of-function mutations in mTORC1 did not. Although the genetic status of the patients was not known, it is possible that some patients had mTORC1 mutations that would not result in increased FLNA expression. The lack of effect of hyperactive mTORC1 on FLNA expression is consistent with our previous observations that increased FLNA expression was due to increased transcriptional activity downstream of hyperactive MEK/MAPK independently of mTORC1. In Rheb^{CA} mice modeling the pathology seen in TSC and FCDII, FLNA expression was also increased in Rheb^{CA} neurons and the cortex containing Rheb^{CA} neurons.

Using an shRNA against Flna, it was found that decreasing FLNA expression in Rheb^{CA} neurons alleviated their misplacement and dysmorphogenesis, including increased soma size and dendritic complexity. The rescue of soma size was unanticipated because knocking down FLNA did not decrease mTORC1activity, which is well-known to regulate cell size. The mechanism of FLNA's role in cell size regulation is not known: FLNA could affect actin cross-linking and/or the activity of many binding partners, including previously unrecognized partners in the hyperactive mTORC1 condition. Identifying how knocking down FLNA alters soma size is outside the scope of the present study. Nevertheless, finding that normalizing FLNA expression did not rescue mTORC1 hyperactivity highlights that FLNA acts via a different mechanism than the mTORC1 blocker, rapamycin, to alleviate cellular abnormalities in TSC and FCDII conditions. Ultimately, normalizing FLNA expression using Flna shRNA in Rheb^{CA}-expressing mice led to a decrease in seizure activity (by 83%) compared to control. This finding seems to contradict the fact that mTORC1 activity is required for epilepsy since rapamycin blocks seizure activity and is also sufficient for epilepsy since patients with FCDII with gain-of-function mTORC1 mutations display seizures and expressing plasmids encoding hyperactive mutant mTORC1 in mice leads to seizures. One possible explanation for this discrepancy is that FLNA interferes with the translation machinery downstream of mTORC1, possibly through cytoskeletal reorganization (considering that poly (A) mRNA colocalized with FLNA or its interaction with disease-specific binding partners regulating cap-dependent translation. FLNA is indeed an actin-cross linking molecule, has multiple binding partners, and acts as a scaffolding platform inside cells where molecules can interact. Considering that increased soma size and dendritic tree require increased translation, a partial reduction of these defects by normalizing FLNA further suggests an interaction between FLNA and translation. This putative function of FLNA should be investigated in future studies. Finally, although the parallel rescue of some of the morphological abnormalities and seizure activity by Flna shRNA implies that these abnormalities contribute to epileptic seizures, it is possible that these rescues are coincidental and rescuing other intracellular processes by Flna shRNA may prevent seizure generation. Identifying how FLNA regulates cellular abnormalities and ultimately epilepsy, perhaps by regulating binding partners and translation is a direction for future studies. Collectively, these data point to a critical role of FLNA in the generation of FCM and epileptic seizures.

Next was examined the small molecule PTI-125, which has been reported to bind aberrant FLNA in Alzheimer's disease brain. Similar to Flna shRNA, it was found that PTI-125 ameliorated neuronal dysmorphogenesis, including increased soma size and dendritic complexity in mice treated from P8 to P28. PTI-125 treatment after P8 did not rescue neuronal placement, which is normally complete by P8. This finding suggests that the environment is not permissive for later migration and/or that once neurons have entered dendritic development (>P8), migration cannot be reinitiated. Mice treated with PTI-125 starting during the neonatal period displayed reduced seizure frequency compared to littermate mice treated with vehicle (saline) during the same period. The fact that neuronal misplacement was not rescued whereas seizure activity was decreased by PTI-125 is in agreement with a previous report that misplacement is not required for seizure activity. This finding also shows that alleviating seizure activity can be achieved even if neuronal misplacement is not rescued. Considering that most patients with epilepsy would be treated after the onset of seizures, also examined were the effects of PTI-125 on seizures after mice had experienced seizures for one week or for more than a month. Convulsive seizures are visible at three weeks of age. Treatment of juvenile mice with PTI-125 at 29 (about one week after seizure onset) partially normalized soma size and dendritic abnormalities, and reduced seizure frequency (by 67%). Mice treated with PTI-125 starting at P61 (>one month after seizure onset) and recorded prior to and during treatment exhibited a progressive decrease in seizure frequency over time, reaching a 60% decrease after 30 days of treatment. By contrast, seizure activity in saline-treated mice increased over time.

In conclusion, targeting FLNA with shRNA gene therapy or the small molecule PTI-125 may offer an alternative option for treating seizures and epilepsy in patients with TSC or selective FCDII without altering mTORC1 activity. These treatment options would likely not be applicable to all patients with FCD, but those with mutations in PI3K-Rheb pathway genes. The proposed treatment would nevertheless be applicable to all TSC individuals. The findings presented here suggest the possibility of combination therapy with mTORC1 blocker, everolimus (a rapamycin derivate), perhaps at lower and less adverse doses. A more thorough study of dosing and duration of treatment might identify a regimen to enhance PTI-125's efficacy in treating seizures. Clinical development of PTI-125 for another indication (Alzheimer's disease) is already underway, which should greatly facilitate clinical application in patients with TSC and FCDII with mutations in PI3K-Rheb pathway genes.

### Example 6

Overview: KGN efficacy on neuron morphology and seizure activity using video-EEG recordings will be tested. Two different doses of KGN on seizure activity will be tested. The doses to be tested will be determined by ore-testing the effect of different KGN doses on neuronal morphology.

### Research Design:

For morphology: mice expressing Rheb^{CA} in the medial prefrontal cortex (mPFC) will be generated. Mice will be treated with KGN at different doses from P8 to P28. Then neuronal soma size and dendritic complexity and length will be analyzed at P28.

For seizures: Mice expressing Rheb^{CA} in the medial prefrontal cortex (mPFC) that have convulsive seizures will also be generated. A 2-3 μg/μl concentration of Rheb^{CA} that was used for data in FIGS. 3E-3G will be used. Mice will be pre-screened for seizure activity by visual observation. Seizures will be routinely observe by 21 days of age. Mice will then undergo EEG recording and video-monitoring 24/7 starting at 6-8 weeks.

Experiment 1: Mice will be randomly split into two groups: one that receives vehicle and another one that receives KGN used at the dose that was the most efficient at rescuing neuronal morphology. KGN will be given from P28 for 4 weeks. Seizure activity will be recorded for the last week. Based on past experience and power analysis, 37pproximately 15-20 mice will be needed to reach a significant 50% effect size.

Experiment 2: Mice will be recorded for 6 weeks. Mice will receive vehicle for the first 2 weeks, followed by KGN injections. Control mice will receive vehicle throughout the recordings. 37pproximately 10-15 mice will be needed to reach a significant 50% effect size.

Analysis of KGN efficacy: Several types of analyses will be performed. First a Friedman test will be used followed by Dunn's test. The seizure frequency will also be averaged for the last 2 weeks of recordings and a paired t-test statistical analysis will be performed.
Brain pathology analysis: Soma size post-EEG recordings will be quantified for all the mice studied.
Expected results: It is aniticipated that KGN significantly normalizes neuronal morphology completely or partially and significantly decreases seizure frequency.

TABLE 1
Information for patients with FCDII

| | | | | | | | |
|---|---|---|---|---|---|---|---|
| Patient | Relative | Age at | Duration of | | Location of | | |
| # FCD | FLNA | surgery | epilepsy | | surgical | | Antiseizure |
| type | quantity | (yrs) | history | Gender | resection | Pathology | drugs |

Immunostaining

| | | | | | | | | |
|---|---|---|---|---|---|---|---|---|
| 1 | N/A | 2 | 1 | year | M | L superior | Cortical | Topiramate, |
| FCD | | | | | | frontal gyrus | dyslamination, | oxcarbazepine, |
| Iib | | | | | | | dysmorphic neurons, | lev |
| | | | | | | | balloon cells, and | |
| | | | | | | | gliosis | |
| 2 | N/A | 5 | 3 | years | M | R supramarginal | Cortical | Oxcarbazepine |
| FCD | | | | | | gyrus | dyslamination, | |
| Iib | | | | | | | dysmorphic neurons, | |
| | | | | | | | and balloon cells | |
| 3 | N/A | 4 | 2 | months | M | L superior and | Cortical | Lev, sodium |
| FCD | | | | | | middle frontal | dyslamination, | valproate |
| Iib | | | | | | gyrus | dysmorphic neurons, | |
| | | | | | | | and balloon cells; | |
| | | | | | | | IHC: NF-Pan+, | |
| | | | | | | | NeuN+, Syn+, | |
| | | | | | | | Olig2+, GFAP+, | |
| | | | | | | | CD34+ | |
| 4 | N/A | 26 | 6 | years | F | R middle frontal | Cortical | Oxcarbazepine, |
| FCD | | | | | | gyrus | dyslamination, | lev, sodium |
| Iib | | | | | | | dysmorphic neurons, | valproate |
| | | | | | | | and balloon cells; | |
| | | | | | | | IHC: NeuN+, | |
| | | | | | | | GFAP+, Olig2+, | |
| | | | | | | | CgA+ | |
| 5 | N/A | 8 | 7 | years | F | L superior | Cortical | Oxcarbazepine, |
| FCD | | | | | | frontal gyrus | dyslamination, | sodium |
| Iia | | | | | | | dysmorphic neurons, | valproate |
| | | | | | | | and immature | |
| | | | | | | | neurons; IHC: | |
| | | | | | | | NeuN+, GFAP+, | |
| | | | | | | | Olig2+, Ki67 (<1%+), | |
| | | | | | | | p53−, MGMT−, | |
| | | | | | | | IDH−, CD34− | |

Immunoblotting

| | | | | | | | | |
|---|---|---|---|---|---|---|---|---|
| 1 | 2.24 | 30 | 5 | years | M | L temporal | Cortical | Carbamazepine |
| FCD | | | | | | | dyslamination, | |
| Iia | | | | | | | dysmorphic neurons, | |
| | | | | | | | and gliosis | |
| 2 | 1.38 | 16 | 2 | years | M | R occipital | Cortical | Lev, |
| FCD | | | | | | | dyslamination and | oxcarbazepine |
| Iia | | | | | | | dysmorphic neurons | |
| 3 | 2.31 | 7 | 7 | years | F | R superior and | Cortical | Lev, |
| FCD | | | | | | middle frontal | dyslamination and | phenobarbital, |
| Iia | | | | | | gyrus | dysmorphic neurons | sodium valproate, |
| | | | | | | | | oxcarbazepine, |
| | | | | | | | | phenytoin, |
| | | | | | | | | clonazepam, |
| | | | | | | | | lamotrigine, |
| | | | | | | | | topiramate |
| 4 | 1.51 | 5 | 3 | years | M | L frontal | Cortical | Carbamazepine, |
| FCD | | | | | | | dyslamination and | oxcarbazepine, |
| Iia | | | | | | | dysmorphic neurons; | lev, topiramate, |
| | | | | | | | IHC: NF-Pan+, | sodium valproate |
| | | | | | | | NeuN+, GFAP+, | |
| | | | | | | | CD34−, CgA+ | |
| 5 | 2.26 | 9 | 1 | month | M | L middle frontal | Cortical | Phenobarbital, |
| FCD | | | | | | gyrus | dyslamination, | oxcarbazepine |
| Iia | | | | | | | dysmorphic neurons | |
| 6 | 1.54 | 11 | 3 | years | F | R occipital | Cortical | Lev |
| FCD | | | | | | | dyslamination, | |
| Iia | | | | | | | dysmorphic neurons | |
| 7 | 1.04 | 15 | 12 | years | F | R angular gyrus | Cortical | Carbamazepine, |
| FCD | | | | | | and | dyslamination, | topiramate |
| Iib | | | | | | supramarginal | dysmorphic neurons, | |
| | | | | | | gyrus | balloon cells, and | |
| | | | | | | | gliosis | |
| 8 | 0.97 | 12 | 8 | years | M | L insular lobe | Cortical | Lamotrigine, |
| FCD | | | | | | | dyslamination and | topiramate |
| Iia | | | | | | | dysmorphic neurons | |
| 9 | 0.57 | 26 | 14 | years | F | R orbitofrontal | Cortical | Magnesium |
| FCD | | | | | | gyrus and gyrus | dyslamination, | valproate, lev, |
| Iib | | | | | | rectus | dysmorphic neurons, | phenobarbital |
| | | | | | | | and balloon cells; | sodium bromide, |
| | | | | | | | IHC: NeuN+, | anti-triazine |
| | | | | | | | GFAP+, CgA+, | |
| | | | | | | | Olig2+, Ki67− | |
| 10 | 1.76 | 29 | 6 | years | M | L frontal and | Cortical | Sodium valproate, |
| FCD | | | | | | cingulate gyrus | dyslamination, | carbamazepine |
| Iib | | | | | | | dysmorphic neurons, | |
| | | | | | | | balloon cells and | |
| | | | | | | | immature neurons. | |
| 11 | 1.97 | 10 | 1 | year | M | L frontal | Cortical | Oxcarbazepine |
| FCD | | | | | | | dyslamination, | |
| Iib | | | | | | | dysmorphic neurons, | |
| | | | | | | | balloon cells, and | |
| | | | | | | | gliosis | |
| 12 | 2.09 | 11 | 10 | years | M | R temporal, | Cortical | Carbamazepine |
| FCD | | | | | | amygdala, | dyslamination, | |
| Iib | | | | | | hippocampus | dysmorphic neurons, | |
| | | | | | | | and balloon cells. | |

TABLE 2
Information for control patients

| Age at | Relative | | | | |
|---|---|---|---|---|---|
| surgery | FLNA | | Location of | | |
| (yrs) | quantity | Gender | resection | Causes | Surgical Diagnosis |
| 13 | 0.417 | F | Right ® | Fall from | R temporal brain contusion |
| | | | temporal | height | |
| 13 | 0.73 | M | R occipital | Fall from | R occipital brain contusion |
| | | | | height | |
| 16 | 1.71 | M | Left (L) | Fall from | L occipital brain contusion, |
| | | | occipital | height | Subdural hemorrhage, basilar |
| | | | | | skull fracture |
| 5 | 1.31 | M | L frontal | Car accident | L frontal brain contusion, diffuse |
| | | | | | axonal injury, frontal bone |
| | | | | | fracture |
| 9 | 1.78 | M | R occipital | Car accident | R occipital brain contusion, |
| | | | | | occipital bone fracture |
| 6 | 0.87 | M | R frontal | Fall from | R frontal brain contusion, |
| | | | | height | subarachnoid hemorrhage, |
| | | | | | frontal bone fracture |
| 23 | 0.52 | M | R parietal | Fall from | R parietal Brain contusion, |
| | | | | height | parietal bone fracture |
| 27 | 0.27 | F | Bifrontal | Car accident | Bifrontal Brain contusion, |
| | | | | | bifrontal bone fracture, L |
| | | | | | temporal bone fracture |
| 10 | 1.11 | M | R frontal | Fall from | R frontal brain contusion, |
| | | | | height | epidural hemorrhage, |
| | | | | | subarachnoid hemorrhage |
| 11 | 1.06 | F | L frontal | Car accident | L frontal brain contusion, |
| | | | | | subarachnoid hemorrhage, |
| | | | | | frontal bone fracture |
| 15 | 1.22 | M | R temporal | Car accident | R frontal brain contusion, |
| | | | | | subarachnoid hemorrhage, |
| | | | | | diffuse axonal injury |

None of the patients had a history of drug.

TABLE 3
Summary of statistical tests

| Fig. | Tests | Statistical values | P values | n | Definition of n |
|---|---|---|---|---|---|
| 1G | Student's t-test, | t = 7.007 df = 99 | <0.0001 | 15 control | Number of cells |
| | unpaired, two- | | | and 19 | from 3 human |
| | tailed | | | SMI-311+ | samples |
| 1I | Student's t-test, | t = 3.121 df = 22 | 0.005 | 12 | Number of human |
| | unpaired, two- | | | | samples per |
| | tailed | | | | condition |
| 2B | One way | F (3, 8) = 46.84 | <0.0001 | 3 | Number of |
| | ANOVA with | | | | neuronal culture |
| | Tukey post-test | | | | sets per condition |
| 2C | One way | F (3, 8) = 73.46 | <0.0001 | 3 | Number of |
| | ANOVA with | | | | neuronal culture |
| | Tukey post-test | | | | sets per condition |
| 2F | Student's t-test, | t = 25.6 df = 34 | <0.0001 | 35 | Number of cells in |
| | paired, two- | | | | 4 mice per |
| | tailed | | | | condition |
| 2G | Student's t-test, | t = 3.058 df = 4 | 0.0377 | 5 | Number of cortices |
| | paired, two- | | | | |
| | tailed | | | | |
| 3A | One way | F (2, 98) = 682.5 | 0.0001 | 30/35/36 | Number of slices in |
| | ANOVA with | | | | 6 mice per |
| | Tukey post-test | | | | condition |
| | | | | | (GFP/Rheb^{CA} + Luc |
| | | | | | shRNA/Rheb^{CA} + |
| | | | | | Flna shRNA) |
| 3B | One way | F (2, 132) = 356.5 | <0.0001 | 45 | Number of cells |
| | ANOVA with | | | | per condition (3 |
| | Tukey post-test | | | | slices/mouse, 3 |
| | | | | | mice) |
| 3C | Two-way | Interaction: | 0.05 | 25-21-26 | Number of cells in |
| | repeated | F (40, 1380) = 10.31, | | | 3 mice per |
| | measures | p < 0.0001 | | | condition |
| | ANOVA | Row factor: | | | (GFP-Rheb^{CA} + Luc |
| | Bonferroni | F (20, 1380) = 294.9, | | | shRNA-Rheb^{CA} + |
| | post-test | p < 0.0001 | | | Flna shRNA) (3 |
| | | Column factor: | | | slices per mouse) |
| | | F (2, 69) = 18.58, | | | |
| | | p = 0.0219 | | | |
| | | Subjects (matching) | | | |
| | | F (69, 1380) = 10.78, | | | |
| | | p < 0.0001 | | | |
| 3D | One way | F (2, 68) = 27.29 | <0.0001 | 25-21-26 | Number of cells in |
| | ANOVA with | | | | 4 mice per |
| | Tukey post-test | | | | condition (GFP- |
| | | | | | Rheb^{CA} + LMc |
| | | | | | shRNA-Rheb^{CA} + |
| | | | | | Flna shRNA) (3 |
| | | | | | slices per mouse) |
| 3G | Mann Whitney U | U = 2 | <0.0001 | 11-10 | Number of mice |
| | test, two-tailed | | | | per condition |
| | | | | | (Rheb^{CA} + Luc |
| | | | | | shRNA-Rheb^{CA} + |
| | | | | | Flna shRNA) |
| 3H | Student's t-test, | t = 0.2668 df = 58 | 0.7905 | 30 | Number of cell |
| | unpaired, two- | | | | doublets (ratio |
| | tailed | | | | GFP+/GFP−) in 4 |
| | | | | | mice per condition |
| 4B | One way | F (3, 181) = 75.52 | <0.0001 | 46 | Number of cells in |
| | ANOVA with | | | | 4 mice per |
| | Tukey post-test | | | | condition |
| 4D, | Two-way | Rheb^{CA} + vehicle vs | 0.05 | 47-35 | Number of cells in |
| left | repeated | Rheb^{CA} + PTI-125 | 0.05 | 47-31 | 6 mice per |
| | measures | Interaction: | | | condition (vehicle |
| | ANOVA | F (30, 2400) = 20.5, | | | vs PTI-125) |
| | Bonferroni | p < 0.0001 | | | |
| | post-test | Row factor: | | | |
| | | F (30, 2400) = 446.4, | | | |
| | | p < 0.0001 | | | |
| | | Column factor: | | | |
| | | F (1, 80) = 45.3, | | | |
| | | p = 0.0219 | | | |
| | | Subjects (matching) | | | |
| | | F (80, 2400) = 11.91, | | | |
| | | p < 0.0001 | | | |
| | | Rheb^{CA} + vehicle vs | | | |
| | | Rheb^{CA} + PTI-125 | | | |
| | | Interaction: | | | |
| | | F (30, 1950) = 23.12, | | | |
| | | p < 0.0001 | | | |
| | | Row factor: | | | |
| | | F (30, 1950) = 401.5, | | | |
| | | p < 0.0001 | | | |
| | | Column factor: | | | |
| | | F (1, 65) = 58.75, | | | |
| | | p = 0.0219 | | | |
| | | Subjects (matching) | | | |
| | | F (65, 1950) = 8.781, | | | |
| | | p < 0.0001 | | | |
| 4D, | One way | F (3, 139) = 58.05 | <0.0001 | 32-47-31-35 | Number of cells in |
| right | ANOVA with | | | | 6 mice per |
| | Tukey post-test | | | | condition |
| | | | | | (GFP + vehicle- |
| | | | | | Rheb^{CA} + vehicle- |
| | | | | | Rheb^{CA} + 6mg/kg |
| | | | | | PTI-125-Rheb^{CA} + |
| | | | | | 12 mg/kg PTI-125 |
| 4E | Student's t-test, | t = 0.4713 df = 12 | 0.6459 | 6 | Number of cortices |
| | unpaired, two- | | | | per condition |
| | tailed | | | | |
| 4G | Mann Whitney U | U = 1 | 0.0031 | 5-8 | Number of mice |
| | test, two-tailed | | | | per condition |
| | | | | | (Rheb^{CA} + vehicle- |
| | | | | | Rheb^{CA} + PTI-125) |
| 4H | Student's t-test, | t = 0.5367 df = 7 | 0.6081 | 4-5 | Number of mice |
| | unpaired, two- | | | | per condition |
| | tailed | | | | (Rheb^{CA} + vehicle- |
| | | | | | Rheb^{CA} + PTI-125) |
| 4I | Student's t-test, | t = 0.7904 df = 28 | 0.4359 | 15 | Number of cells in |
| | unpaired, two- | | | | 5 mice per |
| | tailed | | | | condition |
| | | | | | (Rheb^{CA} + vehicle- |
| | | | | | Rheb^{CA} + PTI-125) |
| 4J | Student's t-test, | t = 0.3808 df = 58 | 0.7047 | 30 | Number of cells in |
| | unpaired, two- | | | | 5 mice per |
| | tailed | | | | condition |
| | | | | | (Rheb^{CA} + vehicle- |
| | | | | | Rheb^{CA} + PTI-125) |
| 5D | Two-way | Rheb^{CA} + vehicle vs | 0.05 | 21 | Number of cells in |
| | repeated | Rheb^{CA} + PTI-125 | | | 4 mice per |
| | measures | Interaction: | | | condition |
| | ANOVA | F (30, 2400) = 20.5, | | | (Rheb^{CA} + vehicle- |
| | Sidak post-test | p < 0.0001 | | | Rheb^{CA} + PTI-125) |
| | | Row factor: | | | |
| | | F (30, 2400) = 446.4, | | | |
| | | p < 0.0001 | | | |
| | | Column factor: | | | |
| | | F (1, 80) = 45.3, | | | |
| | | p = 0.0219 | | | |
| | | Subjects (matching) | | | |
| | | F (80, 2400) = 11.91, | | | |
| | | p < 0.0001 | | | |
| 5E | One way | F (2, 60) = 53.15 | <0.0001 | 21 | Number of cells in |
| | ANOVA with | | | | 4 mice per |
| | Tukey post-test | | | | condition |
| | | | | | (GFP + vehicle- |
| | | | | | Rheb^{CA} + vehicle- |
| | | | | | Rheb^{CA} + PTI-125) |
| 5G | Mann Whitney U | U = 60.5 | 0.0158 | 15-16 | Number of mice |
| | test, two-tailed | | | | per condition |
| | | | | | (Rheb^{CA} + vehicle- |
| | | | | | Rheb^{CA} + PTI-125) |
| 5J | One way | F (3, 26) = 5.634 | 0.0041 | 7 (vehicle) | Number of mice |
| | ANOVA with | | | 8 (vehicle | per condition |
| | Tukey post-test | | | to PTI-125) | |
| 5K | Wilcoxon test, | | 0.0156 | 7 | Number of mice |
| | two-tailed | | | | receiving vehicle |
| | | | | | and then PTI-125 |
| 6A | Fisher's exact | | 0.0343 | 3/29 | Number of mice |
| | test, two-sided | | | (vehicle) | without/with |
| | | | | 11/32 | seizures per |
| | | | | (PTI-125) | condition |
| 6C | Mann Whitney U | U = 166.5 | <0.0001 | 27-32 | Number of mice |
| | test, two-tailed | | | | per condition |
| | | | | | (Rheb^{CA} + vehicle- |
| | | | | | Rheb^{CA} + PTI-125) |
| S1E | Mann Whitney U | U = 111 | <0.0001 | 125 | Number of cells |
| | test, two-tailed | | | | per condition from |
| | | | | | 5 human samples, |
| | | | | | 25 cells per |
| | | | | | samples from 5 |
| | | | | | randomly selected |
| | | | | | area of interest |
| S1F | Student's t-test, | t = 27.37 df = 248 | <0.0001 | 125 | Number of cells |
| | unpaired, two- | | | | per condition from |
| | tailed | | | | 5 human samples, |
| | | | | | 25 cells per |
| | | | | | samples from 5 |
| | | | | | randomly selected |
| | | | | | area of interest |
| S4A | Student's t-test, | t = 0.7576 df = 10 | 0.4662 | 7 and 5 | Number of FCDIIa |
| | unpaired, two- | | | | and FCDIIb |
| | tailed | | | | |
| S4B | Pearson | R² = 0.03607 | 0.5544 | 12 | Number of FCDII |
| | correlation | | | | |
| S4C | Pearson | R² = 0.301 | 0.0647 | 12 | Number of FCDII |
| | correlation | | | | |
| S5B | Student's t-test, | t = 12.02 df = 10 | <0.0001 | 6 | Number of cultures |
| up | unpaired, two- | | | | per condition |
| | tailed | | | | |
| S5B | Student's t-test, | t = 0.6001 df = 10 | 0.5618 | 6 | Number of cultures |
| below | unpaired, two- | | | | per condition |
| | tailed | | | | |

TABLE 4
Plasmids

| | IUE | |
|---|---|---|
| | concentration | Notes/Origin |
| pCAGGS-Rheb | 1.5 μg/μl | National Institute |
| S16H (i.e., | | of Infectious |
| pCAG-Rheb^{CA}) | | Diseases, Tokyo |
| pCAG- | 1.5 μg/μl | Addgene |
| tdTomato | | (##83029)(19) |
| pCAG-GFP | 1.5 μg/μl | Addgene |
| | | (#11150) |
| pCALNL- | 1.5 μg/μl | Addgene |
| dsRed2 | | (#13769) |
| pCAG-Cre | 0.001 μg/μl | Addgene |
| | | (##13775) |
| pCGLH- | 1.5 μg/μl | The shRNA sequence |
| Flna shRNA | | for FLNA (5′ CCTATG |
| | | AAGCTGGAACCTATA 3′). |
| pCGLH- | 1.5 μg/μl | 5′ CGCTGAGTACTTCG |
| Luciferase | | AAATGTC 3′, |
| shRNA | | sequence from clone |
| | | #TRCN0000072259 |
| pcDNA3-FLAG- | 1 μg/μl | Addgene (#69006) |
| MTOR- | | |
| L1460P | | |
| pcDNA3-FLAG- | 1 μg/μl | Addgene (#69013) |
| MTOR- | | |
| S2215Y | | |
| pcDNA3-EGFP | 1 μg/μl | Addgene (#13031) |

TABLE 5
Primary and Secondary antibodies

| | | | | Concentration |
|---|---|---|---|---|
| | | | | used in IHC or |
| | | Catalog | Host | immunoblot |
| Antibody | Company | Number | animal | (IB) or IF |
| Primary | | | | |
| Filamin A | Abcam | ab51217 | Rabbit | 1:5000 (IB)/ |
| (FLNA) for | | | | 1:500 (IF) |
| IB and IF | | | | |
| Filamin A | Abcam | ab76289 | Rabbit | 1:500 (IHC) |
| (FLNA) for | | | | |
| IHC | | | | |
| SMI-311 | Covance | SMI-311R | Mouse | 1:250 (IF) |
| Phospho-S6 | Cell | #5364 | Rabbit | 1:500 (IHC)/ |
| (S240/244) | Signaling | | | 1:5000 (IB)/ |
| | | | | 1:800 (IF) |
| S6 | Cell | #2217 | Rabbit | 1:5000 (IB) |
| | Signaling | | | |
| Tubulin | Cell | #2146 | Rabbit | 1:5000 (IB) |
| | Signaling | | | |
| ER-81 | Covance | PRB-362C | Rabbit | 1:10,000 (IF) |
| GFP | Aves | GFP-1010 | Chicken | 1:800 (IF) |
| GAPDH | Santa Cruz | sc-25778 | Rabbit | 1:5000 (IB) |
| Rheb | Santa Cruz | sc-6341 | Goat | 1:1000 (IB) |
| ERK1/2 | Santa Cruz | Sc-94 | Rabbit | 1:5000 (IB) |
| DAPI | Life | D1306 | | 1:5000 |
| | Technologies | | | |
| Secondary | | | | |
| HRP- | Cell | 7074 | Donkey | 1:5000 (IB) |
| conjugated | signaling | | | |
| anti-rabbit | | | | |
| HRP- | Thermo | A27014 | Rabbit | 1:2000 |
| conjugated | Fisher | | | |
| anti-goat | Scientific | | | |
| α Mouse IgG | Thermo | A-11004 | Goat | 1:1000 |
| Alexa Fluor | Fisher | | | |
| 568 | Scientific | | | |
| α Mouse IgG | Thermo | A28180 | Goat | 1:1000 |
| Alexa Fluor | Fisher | | | |
| 555 | Scientific | | | |
| α Rabbit IgG | Thermo | A-31572 | Donkey | 1:1000 |
| Alexa Fluor | Fisher | | | |
| 555 | Scientific | | | |
| α Rabbit IgG | Thermo | A-11034 | Goat | 1:1000 |
| Alexa Fluor | Fisher | | | |
| 488 | Scientific | | | |
| α Chicken | Thermo | A-11039 | Goat | 1:1000 |
| IgG | Fisher | | | |
| Alexa Fluor | Scientific | | | |
| 488 | | | | |
| α Rabbit IgG | Thermo | A-31573 | Donkey | 1:1000 |
| Alexa Fluor | Fisher | | | |
| 647 | Scientific | | | |

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. 5 The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be 10 construed to include all such embodiments and equivalent variations.

1. A method of treating epilepsy in a subject in need thereof, the method comprising providing to the subject an effective amount of a FLNA modulator.

2. The method according to claim 1, wherein the FLNA modulator is PTI-125.

3. The method according to claim 1, wherein the epilepsy is intractable epilepsy.

4. The method according to claim 1, wherein the epilepsy is associated with focal cortical dysplasia (FCD) type II or tuberous sclerosis complex (TSC).

5. The method according to claim 1, wherein the FLNA modulator is formulated in a pharmaceutical composition comprising at least one pharmaceutically acceptable excipient.

6. The method according to claim 1, wherein the subject is a mammal.

7. The method according to claim 1, wherein the subject is a human.

8. A method of treating focal cortical dysplasia (FCD) type II or tuberous sclerosis complex (TSC) in a subject in need thereof, the method comprising providing to the subject an effective amount of PTI-125.

9. The method according to claim 1, wherein the FLNA modulator is kartogenin.

10. (canceled)

11. (canceled)

12. The method according to claim 8, wherein the FLNA modulator is formulated in a pharmaceutical composition comprising at least one pharmaceutically acceptable excipient.

13. The method according to claim 8, wherein the subject is a mammal.

14. The method according to claim 8, wherein the subject is a human.

15. A method of inhibiting hyperphosphorylation of the tau protein that comprises the steps of administering to cells of the central nervous system in recognized need, a FLNA-binding effective amount of kartogenin or a pharmaceutically acceptable salt thereof.

16. A method for determining the likelihood of a living patient having Alzheimer's disease pathology (AD pathology) comprising the steps of
a) determining the amount of one or more of a protein-protein complex selected from the group consisting of i) α7nAChR/FLNA, ii) TLR4/FLNA and iii) α7nAChR/Aβ present in a first portion of a lymphocyte preparation from said living patient;
b) determining the amount of said one or more of i) α7nAChR/FLNA, ii) TLR4/FLNA and iii) α7nAChR/Aβ present as a protein-protein complex in a second portion of said lymphocyte preparation, said second portion of said lymphocyte portion further containing admixed therein a FLNA binding-effective amount of kartogenin or a pharmaceutically acceptable salt thereof; and
c) comparing the values so determined, whereby a determined amount of said one or more of i) α7nAChR/FLNA, ii) TLR4/FLNA and iii) α7nAChR/Aβ present as a protein-protein complex in a second portion of said lymphocyte preparation that is significantly decreased in the presence of kartogenin or pharmaceutically acceptable salt thereof indicates that the patient had AD pathology at the time the body sample was taken, whereas no significant difference between the two determined values indicates that the patient was free of AD pathology at the time the body sample was taken.
